SEQUENCE LISTING (16 sequences)

SEQ ID NO: 1 - protein, 13 aa, synthetic construct
  MOD_RES 1: note = Cholic Acid (ChAc)
CGYGPKKKRKVGG

SEQ ID NO: 2 - protein, 7 aa, synthetic construct
PKKKRKV

SEQ ID NO: 3 - protein, 19 aa, synthetic construct
CGWWGYGPKKKRKVGGWWG

SEQ ID NO: 4 - protein, 20 aa, synthetic construct
CSNFGPMKGGNFGGRSSGPY

SEQ ID NO: 5 - protein, 20 aa, synthetic construct
CSGYGKVSRRGGHQNSYKPY

SEQ ID NO: 6 - protein, 20 aa, synthetic construct
CNEKRKEKNIKRGGNRFEPY

SEQ ID NO: 7 - protein, 21 aa, synthetic construct
CADREEGKERRHHRREELAPY

SEQ ID NO: 8 - protein, 34 aa, synthetic construct
CNKRVCEEIAIIPSKKLRNKGSGRIQRGPVRGIS

SEQ ID NO: 9 - protein, 16 aa, synthetic construct
CMGRVRTKTVKKAAGG

SEQ ID NO: 10 - protein, 20 aa, synthetic construct
CNKRVCEEIAIIPSKKLRNK

SEQ ID NO: 11 - protein, 20 aa, synthetic construct
CSKKLRNKIAGYVTHLMKRI

SEQ ID NO: 12 - protein, 15 aa, synthetic construct
CGYGPAAKRVKLDGG

SEQ ID NO: 13 - protein, 22 aa, synthetic construct
CGRFSPMGVDHMSGLSGVNVPG

SEQ ID NO: 14 - protein, 15 aa, synthetic construct
CGYGKLKIKRPVKGG

SEQ ID NO: 15 - protein, 21 aa, synthetic construct
CAVKRPAATKKAGQAKKKKLD

SEQ ID NO: 16 - protein, 8 aa, synthetic construct
SIINFEKL

(19) United States
(12) Patent Application Publication
(10) Pub. No.: US 2025/0051478 A1
(43) Pub. Date: Feb. 13, 2025

(54) scFv and Antibodies with Reduced Multimerisation

(71) Applicant: Y-mAbs Therapeutics, Inc., New York, NY (US)

(72) Inventors: Torben LUND-HANSEN, Roskilde (DK); Nico LIEBENBERG, Fredensborg (DK); Matias Munck MORTENSEN, Roskilde (DK); Steen LISBY, Hørsholm (DK)

(21) Appl. No.: 18/717,536

(22) PCT Filed: Dec. 14, 2022
(86) PCT No.: PCT/DK2022/050280

§ 371 Date: Jun. 7, 2024

(30) Foreign Application Priority Data
Dec. 15, 2021 (DK) PA 2021 70622

(51) Int. Cl. C07K 16/44; C07K 16/10; C07K 16/28; C07K 16/30; C07K 16/32

(52) U.S. Cl. CPC C07K 16/44; C07K 16/1027; C07K 16/2803; C07K 16/2887; C07K 16/2896; C07K 16/3084; C07K 16/32; C07K 2317/31; C07K 2317/565; C07K 2317/622

(57) ABSTRACT

Disclosed are methods for generating variants of scFv, having reduced tendency of forming multimers.

The present invention relates to variants of scFv and antibodies having a reduced tendency of forming multimers. In particular the invention relates to bispecific antibodies comprising said scFvs.

The invention further relates to pharmaceutical compositions comprising one or more antibodies of the invention and the use thereof for treatment of cancer.

### TECHNICAL BACKGROUND

scFv fragments and antibodies, such as bispecific antibodies, have found wide use in antibody assisted diagnosis and therapy.

Pretargeted radioimmunotherapy (PRIT) is one example of a pharmaceutical method that has utilized the efficient binding of antibodies to a specific target allowing treatment to be focused at the targeted site.

WO2018204873 discloses the SADA technology, which benefits from SADA domains having the capability of assembling or disassembling dependent on concentration. This property is particularly beneficial in connection with PRIT. A bispecific antibody capable of binding a cytotoxic agent and a target site, connected to a SADA domain can be administered in multimeric form, in particular tetrameric form, and bind to the antibody target site, whereas unbound molecules will disassemble and be removed from the plasma stream via the kidneys before the cytotoxic agent is administered.

It is important for SADA-PRIT therapies that the molecular size of the multimeric form, in particular tetrameric form, is above the renal clearance limit and thereby providing a long plasma half-life, and that the monomeric form is below the renal clearance limit, providing a short plasma half-life.

For pharmaceutical compositions comprising one or more antibodies it is also important that the one or more antibodies remains stable during the shelf life of the pharmaceutical composition, avoiding degradation of the antibodies as well as unintended agglomeration or cross reactions between antibodies and/or between antibodies and other components of the composition.

### SUMMARY OF THE INVENTION

In a first aspect the invention relates to a method of generating variants of a scFv domain, comprising a light chain variable domain (VL), a heavy chain variable domain (VH) and one or more disulfide bonds between the VL and the VH, comprising the steps of
a. Identifying the cysteine residues forming said one or more disulfide bonds between VL and VH; and
b. Substituting the cysteine residues forming one or more of the disulfide bonds identified in step a., with amino acids different from cysteine.

The variant scFvs of the invention, which are scFvs by themselves, have reduced ability and/or tendency to form multimers compared with scFvs with a disulfide bond between the VH and VL domain.

In another aspect the invention relates to scFv domains prepared according to the method of the invention.

In a further aspect the invention relates to bispecific antibodies comprising a first scFv domain capable of binding DOTA or DOTAM metal chelate, a second scFv domain capable of binding a tumor antigen, and a SADA domain, wherein the first scFv domain and/or the second scFv domain do not contain a disulfide bond between the VH and VL domains.

In a further aspect the invention relates to compositions, in particular pharmaceutical compositions, comprising a scFv or bispecific antibody of the invention, and the use of such compositions for diagnosing or treating cancer.

In a further aspect the invention relates to a kit comprising a scFv or bispecific antibody of the invention.

The invention also relates to polynucleotides, expression vectors or constructs, comprising such polynucleotides, host cell comprising such a polynucleotide or expression vector or constructs and the use of such host cells for the preparation of the scFv or bispecific antibodies on the invention.

Additional aspects are provided in the claims.

### Definitions

DOTA: DOTA (Dodecane Tetraacetic Acid) is also referred to as 1,4,7,10-tetraazacyclododecane-1,4,7 10-tetraacetic acid, and has the formula (CH₂CH₂NCH₂CO₂H)₄ also known as C₁₆H₂₈N₄O₈·xH₂O.
DOTA metal chelate: means DOTA with a complex bound metal ion.
Derivative of DOTA: is intended to mean a compound comprising the DOTA ring system and is capable of chelating metal ions. Examples of such compounds include Benzyl-DOTA and the bispecific chelators disclosed in WO2019010299A. Additional DOTA derivatives are disclosed in WO2010099536 A1.
DOTAM: is a chelator comprising a ring system capable of binding metal ions. It has the systematic name of 1,4,7,10-Tetraazacyclododecane-1,7-bis(acetate)-4,10-bis(acetamide) and has the formula C₁₆H₃₀N₆O₆·2H₂O
Amino acid substitution: is intended to mean the replacement of one amino acid with a different amino acid. In the present specification the term amino acid substitution(s) with reference to a reference sequence is intended to mean that the amino acid sequence in question can be generated starting from the reference sequence and introducing said amino acid substitution(s), even if the sequence in question actually was generated by another process not involving the reference sequence.
Sequence identity: The term Sequence identity is intended to mean a measurement of the relatedness of two nucleic or amino acid sequences. Sequence identity is determined by aligning the two sequences and finding the longest overlap, counting the number of matches in the overlap and calculating the sequence identity by dividing the number of matches by the number of, nucleotide or amino acid, residues in the overlap. Sequence identity is typically expressed in percent (%).
A variety of computational algorithms are available for the skilled person, for generating sequence alignment and calculating Sequence identity. As used herein, Sequence alignment refers to Pairwise alignments. Several algorithms perform this including the sequence alignment program Clustal Omega [doi:10.1038/rnsb.2011.75].
As used herein the sequence alignment are performed using the algorithm:
Algorithm: Clustal Omega (1.2.4),
(http://www.clustal.org/omega/).
Antibody or antibody fragment: An antibody fragment is a portion of an antibody such as F(ab′)2, F(ab)2, Fab′, Fab, Fv, scFv and the like. Regardless of structure, an antibody fragment binds with the same antigen that is recognized by the intact antibody. For example, an 3F8 monoclonal antibody fragment binds with an epitope recognized by 3F8. The term “antibody fragment” also includes any synthetic or genetically engineered protein that acts like an antibody by binding to a specific antigen to form a complex. For example, antibody fragments include isolated fragments consisting of the variable regions, such as the “Fv” fragments consisting of the variable regions of the heavy and light chains, recombinant single chain polypeptide molecules in which light and heavy variable regions are connected by a peptide linker (“scFv proteins”), and minimal recognition units consisting of the amino acid residues that mimic the hypervariable region.
ScFv domain: Single chain polypeptide consisting of the variable regions of the light (VL) and heavy (VH) antibody chains, usually separated by a linker sequence. The order of the VL and the VH regions can vary and it is not unusual that a scFv with the VL-VH order can be changed into a scFv with the VH-VL order without significant change of specificity. The linker sequence separating VL and VH typically comprises, or consist of, small hydrophobic amino acid residues such as G, S and T and may have a size of 5-50 amino acids. One preferred linker consists of four glycine and one serine residues or repeats of such a sequence e.g. 2, 3, 4 or 5 repeats of this sequence.
Substitutions: is understood in the usual way, as a replacement of one amino acid residue in a polypeptide with a different amino acid residue. Substitutions are in this specification and claims described with the format [original amino acid][position][new amino acid], and the single letter code is used. In case that more than one amino acid are possible substituents for a given position the possible substituents are separated by a comma. For example, a substitution of a glycine residue in position 9 of a polypeptide with an alanine residue is noted as G9A, and a substitution of a glycine residue in position 9 with either an alanine or a valine is noted as G9A, V.
Domain: is intended to mean a polypeptide or part of a polypeptide having its own structure and function defined by its amino acid sequences. A polypeptide may be a single domain polypeptide, where the single domain forms the whole polypeptide, or the polypeptide may be a multi domain polypeptide, where the domains are arranged as separate part of the sequence. For multi domain polypeptides linker sequences are often provided between the domains in order to secure a distance between the domains so each domain can be folded and exert its function without steric hinderances from other parts (domains) of the polypeptide.
CDR: Complementarity Determining Regions (CDR) are part of the variable regions of antibodies and are of key importance for the binding specificity of an antibody. A typically antibody consisting of two heavy chains and two light chains has 6 CDR sequences, three in the light chain variable domain (VL) and three in the heavy chain variable domain (VH).
Pharmaceutical composition: As used herein the term “Pharmaceutical composition” is intended to mean a composition for administration as a drug or medicine to a patient in need thereof. Pharmaceutical compositions are prepared from pharmaceutical grade ingredients e.g., as described in European Pharmacopoeia 10^{th} Edition, using methods and technologies known in the pharmaceutical or apothecary area.

### DETAILED DISCLOSURE

Some embodiments of the present invention are provided in the claims.

In one embodiment the invention relates to a method of generating variants of a scFv domain, comprising a light chain variable domain (VL), a heavy chain variable domain (VH) and one or more disulfide bonds between the VL and the VH, comprising the steps of
a. Identifying the cysteine residues forming said one or more disulfide bonds between VL and VH; and
b. Substituting the cysteine residues forming one or more of the disulfide bonds identified in step a., with amino acids different from cysteine.

According to the invention one or all of the disulfide bonds identified in step a., may be removed by substituting the cysteines forming said bond(s) with other amino acids. It is preferred to substitute both cysteines forming a disulfide bond with other amino acids in order to avoid any free cysteine.

The invention is based on the observation that many scFv domains and constructs comprising scFv domains may form multimers, such as dimers or trimers; or multiple forms of the monomer form. This is not desirable for compounds intended for pharmaceutical use, where high uniformity and purity of the compounds are generally desired. Further, heterogenicity of scFv domains and constructs comprising scFv domains complicate recovery and purification compared with similar compounds having a higher homogenicity.

The inventors have realized that disulfide bonds between the VH and VL of the scFv are responsible for multimerization and formation of alternative disulfide bonding leading to the observed formation of multimers and multiple forms of the scFvs, and that scFvs without disulfide bonds between the VH and VL domains have less tendency of forming multimers or multiple forms can be provided using the method of the invention.

The obtained scFvs have similar binding properties as the scFvs from which the variants were derived according to the method of the invention. The skilled person will further realize that the variants derived from a scFv according to the invention are in fact also a scFv in itself.

In natural antibodies the VL and VH sequences are part of the Light and Heavy immunoglobulin chains and in nature the light and heavy chains are connected by one or more disulfide bonds found in the constant regions adjacent to the VL and VH sequences. However, since a scFv consists of only the VL and VH sequences connected by a linker, the disulfide bonds, found in the constant regions adjacent to the VL and VH sequences, and which in natural antibodies connects the chains containing the VL and VH chains, are not present in scFvs and it is therefore common practice to introduce a disulfide bond in scFvs, between the VL and VH sequences in order to improve stability of the scFv. The invention is based on the inventor's realization that such an introduced stabilizing disulfide bonds between the VH and VL domain of an scFv may lead to heterogenicity, which may be disadvantageous for at least some uses of the scFv.

It is known in the area that VH and VL domains may comprise additional disulfide bonds between two cysteine residues in the same domain (intradomain disulfide bonds), and the inventors have further realized that these intradomain disulfide bonds, in contrast to interdomain disulfide bonds (between the VL domain and the VH domain) are not important, or at least less important, for the observed heterogenicity.

In one embodiment the invention relates to a method of generating variants of a scFv domain, wherein said variants give rise to less multimer formation compared with the original scFv domain.

In another embodiment the invention relates to the use of an scFv without a disulfide bond between the VH and VL domain in a polypeptide construct comprising the scFv and an additional domain, where the polypeptide construct has low tendency of multimerize or at least less tendency of forming multimers compared with a similar polypeptide having a disulfide bond between the VH and VL domains of the scFv that, except for this additional disulfide bond, has same sequence.

Multimer formation may be detected using techniques known in the art for determining molecular weights for example chromatographic methods.

In one embodiment of the invention, multimer formation is determined by SDS-PAGE gelelectrophoresis.

In one embodiment of the invention the scFv domain is part of a polypeptide comprising additional antibody fragments. For example, the scFv may be part of a polypeptide that in addition to the scFv domain comprises one or more of: additional scFv domains, immunoglobulin heavy and/or light chains, Fc domains, hinge regions etc.

In one preferred embodiment, the scFv domain is part of a bi- or a multispecific antibody.

One form of such a bi- or multispecific antibody is a bispecific antibody comprising two immunoglobulin heavy chains and two chains of a fusion polypeptide comprising an immunoglobulin light chain C-terminally fused to an scFv domain, where a first binding specificity is provided by the variable regions of the immunoglobulin heavy and light chains and a second binding specificity is provided by the scFv domains.

Another form of such a bi- or multispecific antibody is a polypeptide comprising two or more scFv domains, each providing a binding specificity.

In one embodiment, the invention relates to a bi- or multispecific antibody further comprising a SADA domain, also known as a tetramerization domain.

SADA (self assembly and disassembly) domains are short amino acid domains capable of spontaneously assembling and disassembling in solution, depending on concentration. Complexes comprising a SADA domain typically exists in at least two distinct forms, a tetrameric form at high concentration and a monomeric form at low concentration. The self assembly and disassembly (SADA) technology has been disclosed in WO 2018204873A1, which is incorporated in its entirety by reference.

SADA-complexes may be designed so that the tetrameric form has a molecular weight well above the renal clearance limit and the monomeric form has a molecular weight below the renal clearance limit, meaning that the tetrameric form will have a high plasma-half-life, because it is not excreted into the urine, and the monomeric form has a low plasma half-life, because it is excreted into the urine.

Preferred SADA domains for use according to the invention includes domains comprising the sequence of SEQ ID NO: 5, SEQ ID NO: 6, SEQ ID NO: 7, SEQ ID NO: 8, SEQ ID NO: 9, SEQ ID NO: 10, SEQ ID NO: 11 or SEQ ID NO: 12, or a sequence with at least 80%, at least 85%, at least 90%, at least 95%, at least 97% sequence identity to one of these sequences.

Preferred SADA domains for use according to the invention include domains having the sequence of:
a. SEQ ID No. 5 or a sequence that differs from this sequence by 1, 2, 3, 4, 5, 6, 7, 8, 9 or 10 substitutions;
b. SEQ ID No. 6 or a sequence that differs from this sequence by 1, 2, 3, 4, 5, 6, 7, 8, 9 or 10 substitutions;
c. SEQ ID No. 7 or a sequence that differs from this sequence by 1, 2, 3, 4, 5, 6, 7, 8, 9 or 10 substitutions;
d. SEQ ID No. 8 or a sequence that differs from this sequence by 1, 2, 3, 4, 5, 6, 7, 8, 9 or 10 substitutions;
e. SEQ ID No. 9 or a sequence that differs from this sequence by 1, 2, 3, 4, 5, 6, 7, 8, 9 or 10 substitutions;
f. SEQ ID No. 10 or a sequence that differs from this sequence by 1, 2, 3, 4, 5, 6, 7, 8, 9 or 10 substitutions;
g. SEQ ID No. 11 or a sequence that differs from this sequence by 1, 2, 3, 4, 5, 6, 7, 8, 9 or 10 substitutions; or
h. SEQ ID No. 12 or a sequence that differs from this sequence by 1, 2, 3, 4, 5, 6, 7, 8, 9 or 10 substitutions.

Preferred SADA domains according to the invention are domains comprising a sequence with at least 80% sequence identity to amino acids 6-36 of SEQ ID NO: 5, and which differs from the sequence of SEQ ID NO:5 with one or more substitutions, wherein the domain maintains the ability to dimerize or tetramerize.

The skilled person can easily determine whether such a domain with a given substitution maintains the ability to dimerize or tetramerize by simple routine experimentation, or find such information in the literature, e.g. in J. Gencel-Augusto and G-Lozano; Genes & Development 34:1128-1146, incorporated by reference.

A preferred SADA domain according to the invention is a domain with an amino acid sequence that differs from the sequence of amino acids 6-36 of SEQ ID NO: 5 by 1, 2, 3, 4 or 5 substitutions selected among following substitutions:
E6V, Q, K, G, D or A;
Y7S, N, H, F, D or C;
F8Y, V, S, L, I or C;
T9S, P, N or A;
L10V, I or F;
Q11R, L, K, H or E;
I12V, T, M, L or F;
R13S, P, L, H, G or C;
G14W, R or A;
R15S, P, L, H, G or C;
E16V, Q, K, G, D or A;
F18Y, V, S, L, I or C;
E19V, Q, K, G, D or A;
M20V, T, R, L, K or I;
F21L or I;
R22L or G;
E23V, Q, K, G, D or A;
L24M;
N25S, I or D;
E26V, Q, K, G, D or A;
A27V, T, S, G or D;
L28W, V, M or F;
E29Q, G or D;
L30V, R, I, H or F;
K31T, R, Q, N, M or E;
D32Y, V, N, H, G or A;
A33V, T, S, P, G or D;
Q34R, L, K, H or E;
   using the numbering of SEQ ID NO: 5.

The L24P substitution abolished tetramerization completely and should not be applied.

The p53 tetramerization domain comprising the sequence of amino acids 6-36 of SEQ ID NO: 5, is a preferred SADA domain.

The present invention is particular useful in connection with the SADA technology, using a construct comprising one or more scFvs and a SADA domain constructed so a tetrameric complex comprising four such monomers have a size above the renal clearance limit whereas the monomers of such complex have a size below the renal clearance limit. Typically, such a construct comprising one or more scFvs and a SADA domain has a molecular weight of about 50-60 kD in the monomeric form. The complex will after administration in its tetrameric form bind to its target and unbound complexes will disassemble in plasma and be rapidly excreted because its size is below the renal clearance limit. However, if part of the polypeptides form multimers mediated by disulfide bonds between the VH and VL of the scFv, theoretically and depending on the actual construct, the clearance of disassembled complexes may be less efficient because of the formed multimers.

It is therefore particular beneficial to use the present invention in connecting with the SADA technology to reduce the formation of multimers.

In one embodiment the invention relates to a scFv domain comprising a VL and a VH and capable of binding an antigen, wherein the scFv is obtainable according to the method of any of the preceding claims. Preferably, the VH and VL are not connected by any disulfide bond.

In one embodiment the scFv domain of the invention further comprises a linker between the VH and VL. Linkers also sometimes known as spacers are short amino acid sequences created to separate multiple domains in a single protein. Linkers are known in the art and the present invention is not limited to any particular sequence of the linkers. In general, the purpose of linkers is to connect and/or separate different elements of the complex and are typically mainly composed of small hydrophilic amino acids such as glycine, serin and threonine.

In one embodiment the invention relates to an antibody or antibody fragment capable of binding DOTA metal chelate, and comprises:
6 CDR sequences consisting of the sequences of SEQ ID NO: 44-49 or sequences that differs by 1 or 2 substitutions from the sequences of SEQ ID NO: 44-49;
a VL sequence comprising the sequence of SEQ ID NO: 1 or a sequence with at least 90%, 95%, 96%, 97%, 98% or 99% sequence identity to SEQ ID NO: 1; and
a VH sequence comprising the sequence of SEQ ID NO: 2 or a sequence with at least 90%, 95%, 96%, 97%, 98% or 99% sequence identity to SEQ ID NO: 2;
   wherein the amino acids corresponding to position 111 of SEQ ID NO: 1 and position 45 of SEQ ID NO: 2, are not cysteines.

The antibodies and antibody fragments capable of binding DOTA may be derived from the murine antibody 2D12.5 that was affinity matured to increase the affinity to DOTA (WO 2010/099536). When a scFv was generated based on 2D12.5, a stabilizing disulfide bond was generated by inserting cysteines in positions corresponding to position 111 and 179 of SEQ ID NO: 3. This disulfide stabilized scFv was affinity matured in several rounds generating a number of modified antibodies having improved affinity for DOTA-metal, including the scFv named C825 (SEQ ID NO: 3). The inserted disulfide bond appears to have been considered essential because WO 2010/099526 explains that all variants that emerged from the last affinity maturation were discarded because they had lost the stabilizing disulfide bond.

C825 has later been frequently used, and it has been humanized in order to obtain an antibody having excellent DOTA-metal binding properties, giving rise to fewer adverse reactions when administered to humans, and it appears that the disulfide bond located between position 111 and 179 in the corresponding mC825 scFv SEQ ID NO: 3 invariable have been maintained.

Now the present inventors have surprisingly discovered that the disulfide bond between position 111 and 179 is not mandatory to obtain a functional antibody, in fact it is advantageous to remove this disulfide bond including the cysteines corresponding to positions 111 and 179 of mC825 scFv (SEQ ID NO 3).

The fact that the scFv is capable of binding DOTA metal chelate is intended to mean that the scFv is capable of specifically binding DOTA metal chelate, in particular DOTA binding ¹⁷⁵Lu, with a binding constant Kd of about 10⁻⁴ M or less, e.g., in the range of 10⁻⁴M to 10⁻¹² M, e.g., in the range of 10⁻⁵M to 10⁻¹⁰ M, e.g., in the range of 10⁻⁶M to 10⁻⁹ M.

In one embodiment, the scFv of the invention comprises VL and VH domains consisting of SEQ ID NO: 1 and 2.

In one embodiment, the scFv comprising or consisting of the sequence of SEQ ID NO: 4, or comprising or consisting of a sequence having at least 90% sequence identity, e.g. at least 95% sequence identity, e.g. at least 96% sequence identity, e.g. at least 97% sequence identity, e.g. at least 98% sequence identity or at least 99% sequence identity to SEQ ID NO: 4.

In one embodiment the scFv domain of the invention, is capable of binding GD2, and comprises:
6 CDR sequences consisting of the sequences of SEQ ID NO: 13-18, or sequences that differs by 1 or 2 substitutions from the sequences of SEQ ID NO: 13-18;
a VL sequence comprising the sequence of SEQ ID NO: 19 or a sequence with at least 90%, 95%, 96%, 97%, 98% or 99% sequence identity to SEQ ID NO: 19; and
a VH sequence comprising the sequence of SEQ ID NO: 20 or a sequence with at least 90%, 95%, 96%, 97%, 98% or 99% sequence identity to SEQ ID NO: 20;
   wherein the amino acids corresponding to position 97 of SEQ ID NO: 19 and position 44 of SEQ ID NO: 20, are not cysteines.

The fact that the scFv is capable of binding GD2 is intended to mean that the scFv is capable of specifically binding GD2 with a binding constant Kd of about 10⁻⁴ M or less, e.g., in the range of 10⁻⁴M to 10⁻¹² M, e.g., in the range of 10⁻⁵M to 10⁻¹⁰ M, e.g., in the range of 10⁻⁶M to 10⁻⁹ M.

In one embodiment, the scFv domain of the invention comprises or consists of the sequence of SEQ ID NO: 21, or comprises or consists of a sequence having at least 90% sequence identity, e.g. at least 95% sequence identity, e.g. at least 96% sequence identity, e.g. at least 97% sequence identity, e.g. at least 98% sequence identity or at least 99% sequence identity to SEQ ID NO: 21.

In one embodiment the scFv domain of the invention, is capable of binding CD38, and comprises:
6 CDR sequences consisting of the sequences of SEQ ID NO: 22-27 or sequences that differs by 1 or 2 substitutions from the sequences of SEQ ID NO: 22-27;
a VL sequence comprising the sequence of SEQ ID NO: 28 or a sequence that has at least 90%, 95%, 96%, 97%, 98% or 99% sequence identity to SEQ ID NO: 28; and
a VH sequence comprising the sequence of SEQ ID NO: 29 or a sequence that has at least 90%, 95%, 96%, 97%, 98% or 99% sequence identity to SEQ ID NO: 29;
   wherein the amino acids corresponding to position 100 of SEQ ID NO: 28 and position 44 of SEQ ID NO: 29, are not cysteines.

The fact that the scFv is capable of binding CD38 is intended to mean that the scFv is capable of specifically binding CD38 with a binding constant Kd of about 10⁻⁴ M or less, e.g., in the range of 10⁻⁴M to 10⁻¹² M, e.g., in the range of 10⁻⁵M to 10⁻¹⁰ M, e.g., in the range of 10⁻⁶M to 10⁻⁹ M.

In one embodiment the CDR sequences consists of SEQ ID NO: 22-27.

In one embodiment, the scFv of the invention, comprises or consists of the sequence of SEQ ID NO: 30, or comprises or consists of a sequence having at least 90% sequence identity, e.g. at least 95% sequence identity, e.g. at least 96% sequence identity, e.g. at least 97% sequence identity, e.g. at least 98% sequence identity or at least 99% sequence identity to SEQ ID NO: 30.

In one embodiment the scFv domain of the invention, is capable of binding CD20, and comprises:
6 CDR sequences consisting of the sequences of SEQ ID NO: 31-36 or sequences that differs by 1 or 2 substitutions from the sequences of SEQ ID NO: 31-36;
a VL sequence comprising the sequence of SEQ ID NO: 37 or a sequence that has at least 90%, 95%, 96%, 97%, 98% or 99% sequence identity to SEQ ID NO: 37; and
a VH sequence comprising the sequence of SEQ ID NO: 38 or a sequence that has at least 90%, 95%, 96%, 97%, 98% or 99% sequence identity to SEQ ID NO: 38;
   wherein the amino acids corresponding to position 99 of SEQ ID NO: 37 and position 44 of SEQ ID NO: 38, are not cysteines.

The fact that the scFv is capable of binding CD20 is intended to mean that the scFv is capable of specifically binding CD20 with a binding constant Kd of about 10⁻⁴ M or less, e.g., in the range of 10⁻⁴M to 10⁻¹² M, e.g., in the range of 10⁻⁵M to 10⁻¹⁰ M, e.g., in the range of 10⁻⁶M to 10⁻⁹ M.

Preferably, the CDR sequences consists of SEQ ID NO: 31-36.

In one embodiment, the scFv of the invention comprises or consists of the sequence of SEQ ID NO: 39, or comprises or consists of a sequence having at least 90% sequence identity, e.g., at least 95% sequence identity, e.g., at least 96% sequence identity, e.g., at least 97% sequence identity, e.g., at least 98% sequence identity or at least 99% sequence identity to SEQ ID NO: 39.

In one embodiment the scFv domain of the invention is capable of binding GPA33, and comprises:
6 CDR sequences each consisting of the sequences of SEQ ID NO: 50-55 or sequences that differs by 1 or 2 substitutions from the sequences of SEQ ID NO: 50-55;
a VL sequence with the sequence of SEQ ID NO: 56 or a sequence having at least 90% sequence identity, e.g. at least 95% sequence identity, e.g. at least 96% sequence identity, e.g. at least 97% sequence identity, e.g. at least 98% sequence identity or at least 99% sequence identity to SEQ ID NO: 56, wherein the amino acid in position 44 is not a cysteine; and
a VH sequence with the sequence of SEQ ID NO: 57 or a sequence having at least 90% sequence identity, e.g. at least 95% sequence identity, e.g. at least 96% sequence identity, e.g. at least 97% sequence identity, e.g. at least 98% sequence identity or at least 99% sequence identity to SEQ ID NO: 57, wherein the amino acid in position 100 is not a cysteine.

The scFv of this embodiment may comprise or consist of the sequence of SEQ ID NO: 61, or of a sequence having at least 90% sequence identity, e.g. at least 95% sequence identity, e.g. at least 96% sequence identity, e.g. at least 97% sequence identity, e.g. at least 98% sequence identity or at least 99% sequence identity to SEQ ID NO: 61.

In one embodiment the scFv of the invention is capable of binding RSV and comprises
6 CDR sequences consisting of amino acids 26-35, 53-59, 98-109, 177-181, 199-201 and 238-246 of SEQ ID NO: 62 or sequences that differ from these sequences by 1 or 2 substitutions;
a VL sequence with the sequence of amino acids 1-120 of SEQ ID NO: 62 or a sequence having at least 90% sequence identity, e.g. at least 95% sequence identity, e.g. at least 96% sequence identity, e.g. at least 97% sequence identity, e.g. at least 98% sequence identity or at least 99% sequence identity to amino acids 1-120 of SEQ ID NO: 62; and
a VH sequence with the sequence of amino acids 151-256 of SEQ ID NO: 62 or a sequence having at least 90% sequence identity, e.g. at least 95% sequence identity, e.g. at least 96% sequence identity, e.g. at least 97% sequence identity, e.g. at least 98% sequence identity or at least 99% sequence identity to amino acids 151-256 of SEQ ID NO: 62;
   wherein the VL and the VH is not connected by a disulfide bond.

In one embodiment the scFv of the invention is capable of binding B7H3 and comprises
6 CDR sequences consisting of amino acids 26-33, 51-58, 97-107, 175-180, 198-200 and 237-245 of SEQ ID NO: 63 or sequences that differ from these sequences by 1 or 2 substitutions;
a VL sequence with the sequence of amino acids 149-255 of SEQ ID NO: 63 or a sequence having at least 90% sequence identity, e.g. at least 95% sequence identity, e.g. at least 96% sequence identity, e.g. at least 97% sequence identity, e.g. at least 98% sequence identity or at least 99% sequence identity to amino acids 149-256 of SEQ ID NO: 63; and
a VH sequence with the sequence of amino acids 1-118 of SEQ ID NO: 63 or a sequence having at least 90% sequence identity, e.g. at least 95% sequence identity, e.g. at least 96% sequence identity, e.g. at least 97% sequence identity, e.g. at least 98% sequence identity or at least 99% sequence identity to amino acids 1-118 of SEQ ID NO: 63;
   wherein the VL and the VH is not connected by a disulfide bond.

In one embodiment the scFv of the invention is capable of binding HER2 and comprises
6 CDR sequences consisting of amino acids 27-32, 50-52, 89-97, 164-171, 189-196 and 235-247 of SEQ ID NO: 64 or sequences that differ from these sequences by 1 or 2 substitutions;
a VL sequence with the sequence of amino acids 1-108 of SEQ ID NO: 64 or a sequence having at least 90% sequence identity, e.g. at least 95% sequence identity, e.g. at least 96% sequence identity, e.g. at least 97% sequence identity, e.g. at least 98% sequence identity or at least 99% sequence identity to amino acids 1-108 of SEQ ID NO: 64; and
a VH sequence with the sequence of amino acids 138-258 of SEQ ID NO: 64 or a sequence having at least 90% sequence identity, e.g. at least 95% sequence identity, e.g. at least 96% sequence identity, e.g. at least 97% sequence identity, e.g. at least 98% sequence identity or at least 99% sequence identity to amino acid 138-256 of SEQ ID NO: 64;
   wherein the VL and the VH is not connected by a disulfide bond.

In one embodiment the scFv of the invention is capable of binding HER2 and comprises
6 CDR sequences consisting of amino acids 26-33, 51-58, 97-108, 176-181, 199-201 and 238-246 of SEQ ID NO: 66 or sequences that differ from these sequences by 1 or 2 substitutions;
a VL sequence with the sequence of amino acids 150-256 of SEQ ID NO: 66 or a sequence having at least 90% sequence identity, e.g. at least 95% sequence identity, e.g. at least 96% sequence identity, e.g. at least 97% sequence identity, e.g. at least 98% sequence identity or at least 99% sequence identity to amino acids 150-256 of SEQ ID NO: 66; and
a VH sequence with the sequence of amino acids 1-119 of SEQ ID NO: 66 or a sequence having at least 90% sequence identity, e.g. at least 95% sequence identity, e.g. at least 96% sequence identity, e.g. at least 97% sequence identity, e.g. at least 98% sequence identity or at least 99% sequence identity to amino acids 1-119 of SEQ ID NO: 66;
   wherein the VL and the VH is not connected by a disulfide bond.

In one embodiment the scFv of the invention is capable of binding DOTAM and comprises
6 CDR sequences consisting of amino acids 302-310, 327-333, 372-387, 455-462, 480-482 and 519-530 of SEQ ID NO: 68 or sequences that differ from these sequences by 1 or 2 substitutions;
a VL sequence with the sequence of amino acids 429-540 of SEQ ID NO: 68 or a sequence having at least 90% sequence identity, e.g. at least 95% sequence identity, e.g. at least 96% sequence identity, e.g. at least 97% sequence identity, e.g. at least 98% sequence identity or at least 99% sequence identity to amino acids 429-540 of SEQ ID NO: 68; and
a VH sequence with the sequence of amino acids 278-398 of SEQ ID NO: 68 or a sequence having at least 90% sequence identity, e.g. at least 95% sequence identity, e.g. at least 96% sequence identity, e.g. at least 97% sequence identity, e.g. at least 98% sequence identity or at least 99% sequence identity to amino acid 278-398 of SEQ ID NO: 68;
   wherein the VL and the VH is not connected by a disulfide bond.

In one embodiment the invention relates to bispecific antibodies comprising a first and a second binding site, wherein at least one of the first and the second binding sites is a scFv that does not comprise a disulfide bond between the VH and VL domains.

Several forms for bispecific antibodies are known in the art and the present invention is not limited to any particular such form. One example of a bispecific antibody is an antibody comprising two antibody heavy chains and two fusion polypeptides, the fusion polypeptides comprising an antibody light chain where a scFv sequence is fused to the C-terminal of the light chain. Another example of a bispecific antibody is a molecule comprising two or more scFv sequences linked serially after each other.

In one embodiment, the bispecific antibody of the invention comprises a first scFv domain that does not comprise a disulfide bond connecting the VH and the VL and/or a second scFv domain that does not comprise a disulfide bond connecting the VH and the VL.

In one embodiment, the bispecific antibody of the invention further comprises one or more linker sequences.

In one preferred embodiment the invention relates to a bispecific antibody comprising a first scFv domain capable of binding a chelator, a second scFv domain capable of binding a tumor antigen, and a SADA domain, wherein the first scFv domain and/or the second scFv domain do/does not comprise a disulfide bond between the VH and VL domains. Preferably, the first and or the second scFv is a scFv of the invention. The tumor antigen may be any antigen known to be present mainly on the surface of tumor cells, in particular on the surface of solid tumors.

Examples of such tumor antigens include: HER2, B7-H3, CA6, CD138, CD20, CD19, CD22, CD27L, CD30, CD33, CD37, CD38, CD47, CD56, CD66e, CD70, CD74, CD79b, EGFR, EGFRvIII, FRα, GCC, GPNMB, Mesothelin, MUC16, NaPi2b, Nectin 4, PSMA, STEAP1, Trop-2, 5T4, AGS-16, alpha v beta6, CA19.9, CAIX, CD138, CD174, CD180, CD227, CD326, CD79a, CEACAM5, CRIPTO, DLL3, DS6, Endothelin B receptor, FAP, GD2, Mesothelin, PMEL 17, SLC44A4, TENB2, TIM-1, CD98, Endosialin/CD248/TEM1, Fibronectin Extra-domain B, LIV-1, Mucin 1, p-cadherin, peritosin, Fyn, SLTRK6, Tenascin c, VEGFR2, and PRLR.

Preferred examples of tumor antigens include GD2, CD38, B7-H3, CD33, GPA33.

GD2 is a disialoganglioside expressed on tumors of neuroectodermal origin, such as neuroblastoma and melanoma. The expression on normal tissue is highly restricted.

CD38, also known as cyclic ADP ribose hydrolase, is found on the surface of many immune cells including CD4⁺, CD8⁺, B lymphocytes and natural killer cells. The expression is very high on myeloma cells.

B7-H3, B7 homolog 3, also known as CD276 is a type I transmembrane protein that exists in two isoforms. It has limited expression in normal tissue and is expressed at high frequency on many different cancer types e.g. neuroblastoma.

CD33, also known as sialic acid binding Ig-like lectin 3, is a cell surface antigen. It is expressed on cells of myeloid lineage. It can be aberrantly expressed on some cases of plasma-cell myeloma.

GPA33, Glycoprotein 33, is a cell surface antigen that is expressed in greater than 95% of human colon cancers.

The binding site capable of binding a chelator, or a chelator binding a metal ion, may be any such binding site known in the art. Preferred examples of chelators include DOTA, DOTAM, and variants of these. Examples of suitable binding sites capable of binding a chelator or a chelator binding a metal ion may be found in WO 2010/099539, disclosing binding sites capable of binding DOTA or derivatives of DOTA and which binding sites based on the antibody 2D12.5, and WO 2019/201959, disclosing rabbit antibodies capable of binding DOTAM, incorporated by reference.

In one embodiment, the bispecific antibody of the invention further comprises a SADA domain

Preferably the bispecific antibody of the invention comprises:
a. a first scFv binding site capable of binding a chelator;
b. a second scFv binding site capable of binding a tumor antigen; and
c. a SADA domain.

In one example, the bispecific antibody of the invention comprises:
a. a first scFv binding site capable of binding DOTA metal chelate and comprises the VL sequence of SEQ ID NO: 1 and the VH sequence of SEQ ID NO: 2;
b. a second scFv binding site capable of binding a tumor antigen; and
c. a SADA domain.

In another example, the bispecific antibody of the invention comprises:
a. a first scFv binding site capable of binding DOTAM metal chelate and comprises the VL sequence of amino acids 429-540 of SEQ ID NO: 68 and the VH sequence of amino acids 278-398 of SEQ ID NO:68;
b. a second scFv binding site capable of binding a tumor antigen; and
c. a SADA domain.

Preferred examples of bispecific antibodies of the invention include the bispecific antibodies comprising or consisting of one of the sequences SEQ ID NO: 40, SEQ ID NO: 41, SEQ ID NO: 42, SEQ ID NO: 58, SEQ ID NO: 59, SEQ ID NO: 62, SEQ ID NO: 63, SEQ ID NO: 64, SEQ ID NO: 65, SEQ ID NO: 66, SEQ ID NO: 67, SEQ ID NO: 68 or SEQ ID NO: 69.

In one embodiment, the invention relates to a composition comprising a scFv or a bispecific antibody of the invention. Preferably, the composition is a pharmaceutical composition.

In one embodiment the invention relates to the use of a bispecific antibody of the invention, for diagnosing or treating cancer.

The cancer is preferably a solid cancer or tumor.

In one embodiment the invention relates to the use of a bispecific antibody according to the invention in a method comprising the steps:
a. Administering the bispecific antibody to a patient in need thereof; and
b. After a holding period administering DOTA, DOTAM or a derivative thereof;
where the DOTA, DOTAM or derivative thereof binds a radionuclide.

In one embodiment the invention relates to the use of a bispecific antibody comprising a first scFv capable of binding DOTA-metal or DOTAM-metal, a second scFv capable of binding a tumor antigen and a SADA-domain, according to the invention in a method comprising the steps:
a. Administering the bispecific antibody to a patient in need thereof; and
b. After a holding period administering DOTA, DOTAM or a derivative thereof;
where the DOTA, DOTAM or derivative thereof binds a radionuclide.

In this embodiment the bispecific antibody comprising a first scFv capable of binding DOTA-metal, DOTAM-metal or a derivative thereof, a second scFv capable of binding a tumor antigen and a SADA-domain is preferably administered in a tetrameric form.

The holding period should be selected in order to give sufficient time to allow the bispecific antibody to find and bind to tumor antigen and to allow the unbound bispecific antibody in tetrameric form to disassemble into monomeric form and thereby quickly be cleared from the blood stream.

The holding period may be selected in the range of 48-96 hours.

In one embodiment the method further comprises comprising administering a clearing agent after step a and before step b.

In one embodiment of the invention, DOTA or derivative thereof is selected among DOTA, Benzyl DOTA and the bischelate compound

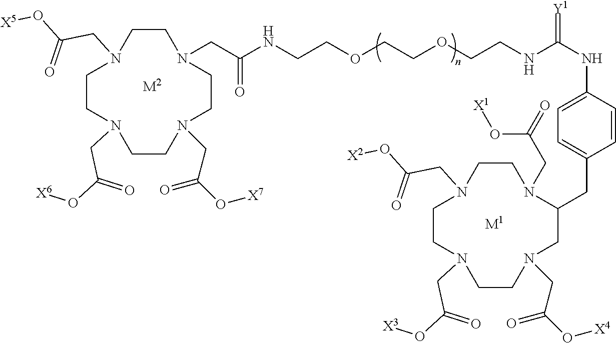

Wherein X¹, X², X³, and X⁴ are each independently a lone pair of electrons (i.e. providing an oxygen anion) or H;
X⁵, X⁶, and X⁷ are each independently a lone pair of electrons (i.e. providing an oxygen anion) or H;
Y¹ is O or S; and
n is 1, 2, 3, 4, 5, 6, 7, 8, 9, 10, 11, 12, 13, 14, 15, 16, 17, 18, 19, 20, 21, or 22; and
M1 is selected among is ¹⁷⁵Lu³⁺, ⁴⁵Sc³⁺, ⁶⁹Ga³⁺, ⁷¹Ga³⁺, ⁸⁹Y³⁺, ¹¹³In³⁺, ¹¹⁵In³⁺, ¹³⁹La³⁺, ¹³⁶Ce³⁺, ¹³⁸Ce³⁺, ¹⁴⁰Ce³⁺, ¹⁴²Ce³⁺, ¹⁵¹Eu³⁺, ¹⁵³Eu³⁺, ¹⁵⁹Tb³⁺, ¹⁵⁴Gd³⁺, ¹⁵⁵Gd³⁺, ¹⁵⁶Gd³⁺, ¹⁵⁷Gd³⁺, ¹⁵⁸Gd³⁺, or ¹⁶⁰Gd³⁺;
M2 is selected among radionuclides.

In one embodiment the radionuclide is selected from the group consisting of ²¹¹At, ⁵¹Cr, ⁵⁷Co, ⁵⁸Co, ⁶⁷Cu, ¹⁵²Eu, ⁶⁷Ga, ¹¹¹In, ⁵⁹Fe, ²¹²Pb, ¹⁷⁷Lu, ²²³Ra, ²²⁴Ra, ¹⁸⁶Re, ¹⁸⁸Re, ⁷⁵Se, ^{99m}Tc ²²⁷Th, ⁸⁹Zr, ⁹⁰Y, ^{94m}Tc, ⁶⁴Cu, ⁶⁸Ga, ⁶⁶Ga, ⁸⁶Y, ⁸²Rb, ^{110m}In, ²⁰⁹Bi, ²¹¹Bi, ²¹²Bi, ²¹³Bi, ²¹⁰Po, ²¹¹Po, ²¹²Po, ²¹⁴Po, ²¹⁵Po, ²¹⁶Po, ²¹⁸Po, ²¹¹At, ²¹⁵At, ²¹⁷At, ²¹⁸At, ²²¹Fr, ²²³Ra, ²²⁴Ra, ²²⁶Ra, ²²⁵Ac, ²²⁷Ac, ²²⁷Th, ²²⁸Th, ²²⁹Th, ²³⁰Th, ²³²Th, ²³¹Pa, ²³³U, ²³⁴U, ²³⁵U, ²³⁶U, ²³⁸U, ²³⁷Np, ²³⁸Pu, ²³⁹Pu, ²⁴⁰Pu, ²⁴⁴Pu, ²⁴¹Am, ²⁴⁴Cm, ²⁴⁵Cm, ²⁴⁸Cm, ²⁴⁹Cf, and ²⁵²Cf, preferable among ¹⁷⁷Lu, ^{99m}Tc, ⁶⁴Cu and ⁸⁹Zr.

The chelator binding a radionuclide, such as DOTA or DOTAM or any derivative thereof bound to a radionuclide; may be administered twice or even more times. When the chelator binding a radionuclide is administered more than once it is recommended that the individual administrations are separated by 24 hours or more.

The two or more administrations of a chelator binding a radionuclide may be using the same radionuclide or, it may be using different radionuclides for each administration.

Such repeated administration of a chelator binding a radionuclide, such as DOTA or a derivative thereof binding a radionuclide; has been disclosed in WO 2021/242848 (incorporated by reference), with respect to GD2-SADA, however, the present inventors have realized that methods using repeated administration of a chelator, such as DOTA; binding a radionuclide is not necessarily limited to GD2-SADA, but can be applied to the bispecific antibodies of the invention.

In one example, a bispecific antibody of the invention, capable of binding a tumor antigen, is administered to a patient in need of such treatment or diagnosis; after 48 hours a chelator binding an alpha-emitter is administered to the patient, 24 hours after the administration of chelator binding the alpha-emitter a second administration of chelator binding a beta-emitter is administered. By using such a method, the benefit of treating using an alpha-emitter and the benefits of a beta-emitter is combined.

In another example, a bispecific antibody of the invention, capable of binding a tumor antigen, is administered to a patient in need of such treatment or diagnosis; after 48 hours a chelator binding a radionuclide suitable for PET or SPECT scanning is administered to the patient and a PET or SPECT scanning is performed. Depending on the outcome of the scanning a treatment procedure can be initiated by administering a chelator binding a radionuclide suitable for treating cancer 24 hours after the first administration of chelator binding a radionuclide, and the treatment may even be continued by administrating a second or subsequent dose of a radionuclide suitable for treating cancer.

Thus, one embodiment the invention relates to the use of a bispecific antibody according to the invention in a method comprising the steps:
a. Administering the bispecific antibody to a patient in need thereof; and
b. After a holding period administering chelator binding a radionuclide,
c. After a further holding period administering chelator binding a radionuclide; and
d. Optionally repeating step c. one or more times.

In one embodiment the method further comprises detecting the localization of the radionuclide. The detection may be performed using well known methods and equipment for detecting radionuclides, such as a PET or SPECT scanner.

In one embodiment the cancer is selected among osteosarcoma, liposarcoma, fibrosarcoma, malignant fibrous histiocytoma, leiomyosarcoma, spindle cell sarcoma, brain tumor, small cell lung cancer, retinoblastoma, HTLV-1 infected T cell leukemia.

The invention further relates to a kit comprising a bispecific antibody according to the invention. Preferably, the kit further comprises chelator, such as DOTA, DOTAM or a derivative of DOTA.

The kit may further comprise instructions for use or a link to such instructions.

The scFv domains and/or the bispecific antibodies of the invention may be prepared using methods known in the art.

One preferred method of providing the scFv domains and/or bispecific antibodies of the invention is to provide a polynucleotide encoding the desired scFv or bispecific antibody, providing the polynucleotide with suitable regulatory sequences, such as promoter, terminator, enhancer, ribosome binding site, Kozak sequence, polyadenylation site etc; inserting the construct in a suitable host cell that subsequently is grown under conditions leading to expression of the desired scFv or bispecific antibody.

The polynucleotide sequence may be assembled using techniques known in art e.g. using PCT technologies or is may be synthesized, for examples using commercial provides for such sequences.

Thus, in one embodiment the invention relates to a polynucleotide encoding a scFv domain or a bispecific antibody of the invention; an expression vector or construct comprising such a polynucleotide sequence, or a host cell comprising the polynucleotide, expression vector or construct.

In one embodiment the invention relates to a method of producing a scFv or a bispecific antibody of the invention, comprising the steps of:
a. Providing a host cell comprising a polynucleotide, an expression vector or construct encoding a scFv or a bispecific antibody of the invention;
b. growing the host cell under conditions inducing expression of the scFv or bispecific antibody; and
c. recovering the scFv or bispecific antibody from the growth broth.

### Sequences

SEQ ID NO: 1: the VL amino acid sequence of the DOTA-metal binding antibody of the invention;
SEQ ID NO: 2: the VH amino acid sequence of the DOTA-metal binding antibody of the invention;
SEQ ID NO: 3: amino acid sequence of mC825;
SEQ ID NO:4: amino acid sequence of huC825 scFv of the invention (without disulfide bond);
SEQ ID NO: 5-12: Amino acid sequences of SADA domains;
SEQ ID NO: 13-18 shows the CDR sequences of GD2 scFv;
SEQ ID NO: 19: VL sequence of the GD2 scFv;
SEQ ID NO; 20: VH sequence of the GD2 scFv;
SEQ ID NO: 21: GD2 scFv without cysteines;
SEQ ID NO:22-27 show the CDR sequences of CD38 scFv;
SEQ ID NO: 28: VL sequence of anti CD38 scFv;
SEQ ID NO: 29: VH sequence of anti-CD38 scFv;
SEQ ID NO: 30 shows the amino acid sequence of anti CD38 without cysteines;
SEQ ID NO: 31-36 show the CDR sequences of anti CD20 scFv;
SEQ ID NO: 37: VL sequence of anti CD20 scFv;
SEQ ID NO: 38: VH sequence of anti CD20 scFv;
SEQ ID NO: 39 shows the amino acid sequence of anti CD20 scFv without cysteines.
SEQ ID NO: 40: shows the amino acid sequence of the GD2-SADA construct without disulfide bonds;
SEQ ID NO: 41: shows the amino acid sequence of the CD38-SADA construct without disulfide bonds;
SEQ ID NO: 42: shows the amino acid sequence of the CD20-SADA construct without disulfide bonds.
SEQ ID NO: 43: shows the amino acid sequence of the GD2-SADA construct with disulfide bonds.
SEQ ID NO: 44-49 show the CDR sequences of C825
SEQ ID NO: 50-55: CDR sequences of anti GPA33
SEQ ID NO: 56: VL sequence of anti GPA33
SEQ ID NO: 57: VH sequence of anti GPA33
SEQ ID NO: 58: GPA33-SADA construct without cysteines
SEQ ID NO: 59: GPA33-SADA construct with cysteines in DOTA scFv
SEQ ID NO: 60: GPA33-SADA construct with cysteines in DOTA scFv and GPA33 scFv
SEQ ID NO: 61: GPA33 scFv without cysteines
SEQ ID NO: 62: shows the amino acid sequence of the RSV-SADA construct without disulfide bonds between the VL and VH sequences. Amino acid 1-120 is the VH sequence of the Anti-RSV scFv, amino acids 151-256 is the VL sequence of the Anti-RSV scFv. The Light chain CDR sequences of the anti-RSV scFv are amino acids 177-181, 199-201 and 238-246 of SEQ ID NO: 62; and the Heavy chain CDR sequences of the anti-RSV scFv are amino acids 26-35, 53-59 and 98-109 of SEQ ID NO: 62.
SEQ ID NO: 63: shows the amino acid sequence of the B7H3-SADA construct without disulfide bonds between the VL and VH sequences. Amino acids 1-118 is the VH sequence of the anti-B7H3, amino acids 149-255 is the VL sequence of the anti-B7H3 scFv. The Light chain CDR sequences of the anti-B7H3 scFv are amino acids 175-180, 198-200 and 237-245 of SEQ ID NO: 63; and the Heavy chain CDR sequences of the anti-B7H3 scFv are amino acids 26-33, 51-58 and 97-107 of SEQ ID NO: 63.
SEQ ID NO: 64: shows the amino acid sequence of the HER2-SADA construct TR-4 (Anti-HER2(VL-VH) x Anti-DOTA (VH-VL)), based on Trastuzumab, without disulfide bonds between the VL and VH sequences. Amino acids 1-108 is the VL sequence of the HER2 scFv, amino acids 138-258 is the VH sequence of the anti-HER2 scFv. The Light chain CDR sequences of the anti-HER2 scFv are amino acids 27-32, 50-52 and 89-97 of SEQ ID NO: 64; and the Heavy chain CDR sequences of the anti-HER2 scFv are amino acids 164-171, 189-196 and 235-247 of SEQ ID NO: 64.
SEQ ID NO: 65: shows the amino acid sequence of the HER2-SADA construct TR-7 (Anti-HER2(VH-VL) x Anti-DOTA (VH-VL)), based on Trastuzumab, without disulfide bonds between the VL and VH sequences. Amino acids 1-120 is the VH sequence of the anti-HER2 scFv, amino acids 151-258 is the VL sequence of the anti-HER2 scFv.
SEQ ID NO: 66: shows the amino acid sequence of the HER2-SADA construct PE-1 (Anti-HER2(VH-VL) x Anti-DOTA (VH-VL)), based on Pertuzumab, without disulfide bonds between the VL and VH sequences. Amino acids 1-119 is the VH sequence of the anti-HER2 scFv, amino acids 150-256 is the VL sequence of the anti-HER2 scFv. The Light chain CDR sequences of the anti-HER2 scFv are amino acids 176-181. 199-201 and 238-246 of SEQ ID NO: 66; and the Heavy chain CDR sequences of the anti-HER2 scFv are amino acids 26-33, 51-58 and 97-108 of SEQ ID NO: 66.
SEQ ID NO: 67: shows the amino acid sequence of the HER2-SADA construct PE-3 (Anti-HER2(VL-VH) x Anti-DOTA (VH-VL)), based on Pertuzumab, without disulfide bonds between the VL and VH sequences. Amino acids 1-107 is the VL sequence of anti-HER2 scFv, amino acids 138-256 is the VH sequence of the anti-HER2 scFv.
SEQ ID NO: 68: shows the amino acid sequence of the CD20-DOTAM-SADA construct Ri-12 (Anti-CD20(VL-VH) x Anti-DOTAM (VH-VL)) without disulfide bonds between the VL and VH sequences. Amino acids 278-398 is the VH sequence of anti-DOTAM scFv; amino acids 429-540 is the VL sequence of anti-DOTAM scFv. The Light chain CDR sequences of the anti-DOTAM scFv are amino acids 455-462, 480-482 and 519-530 of SEQ ID NO: 68; and the Heavy chain CDR sequences of the anti-DOTAM scFv are amino acids 302-310, 327-333 and 372-387 of SEQ ID NO: 68.
SEQ ID NO: 69: shows the amino acid sequence of the CD20-DOTAM-SADA construct Ri-13 (Anti-CD20(VL-VH) x Anti-DOTAM (VL-VH)) without disulfide bonds between the VL and VH sequences. Amino acids 278-389 is the VL sequence of anti-DOTAM scFv, amino acids 420-540 is the VH sequence of the anti-DOTAM scFv.

### FIGURES

FIG. 1 shows a SE-HPLC chromatogram of a GD2-SADA construct. For more details see example 1.

FIG. 2 shows a SE-HPLC chromatogram of a truncated GD2-SADA construct lacking the SADA domain. For more details see example 1.

FIG. 3 shows a Coomassie stained SDS-PAGE gel of the GD2-SADA construct, and the truncated version lacking the SADA domain under non-reducing conditions. The latter is also analyzed under reducing conditions. For more details see example 2.

FIG. 4 shows a Coomassie stained SDS-PAGE gel of variants of a CD20-SADA construct. For more details see example 3.

FIG. 5 shows a Coomassie stained SDS-PAGE gel of variants of a CD38-SADA constructs. For more details see example 4.

FIG. 6 shows a Coomassie stained SDS-PAGE gel of the variant YMS9d under non-reducing or reducing conditions. For more details see example 4.

FIG. 7 shows the dilution regimen and SE-HPLC chromatograms of the diluted samples. For more details see example 6.

FIG. 8 shows Coomassie stained SDS-PAGE gel of the RSV-SADA constructs with or without disulfide bond between the VH and VL sequences. For more details see example 10.

FIG. 9 shows Coomassie stained SDS-PAGE gel of the B7H3-SADA constructs with or without disulfide bond between the VH and VL sequences. For more details see example 11.

FIG. 10A shows Coomassie stained SDS-PAGE gel of the HER2-SADA constructs, based on Trastuzumab, with or without disulfide bond between the VH and VL sequences. For more details see example 12.

FIG. 10B shows Coomassie stained SDS-PAGE gel of the HER2-SADA constructs, based on Pertuzumab, with or without disulfide bond between the VH and VL sequences. For more details see example 12.

FIG. 11 shows Coomassie stained SDS-PAGE gel of the CD20-DOTAM-SADA constructs. For more details see example 13.

All cited references are incorporated by reference.

The accompanying Figures and Examples are provided to explain rather than limit the present invention. It will be clear to the person skilled in the art that aspects, embodiments, claims and any items of the present invention may be combined.

Unless otherwise mentioned, all percentages are in weight/weight. Unless otherwise mentioned, all measurements are conducted under standard conditions (ambient temperature and pressure). Unless otherwise mentioned, test conditions are according to European Pharmacopoeia 8.0.

### EXAMPLES

### Material and Methods

### Production of Proteins:

The nucleotide sequence for the intended protein, including regulatory sequences for directing expression was synthesized and inserted into an expression vector.

The expression vector was transfected into CHO cells and transformants were grown in standard medium for expression of the proteins, whereafter the proteins were recovered from the broth.

### SDS-PAGE Gel Electrophoresis:

Pre-cast gels, Thermo Fisher Bolt Bis-Tris 4-12% gels, were provided from Thermo Fisher Scientific, MA USA, and used according to the manufacturer's instructions. After electrophoresis gels were stained with Coomassie using the manufacturer's instructions.

### Example 1: Disulfide Cross-Linking in GD2-SADA

The GD2-SADA construct with the amino acid sequence SEQ ID NO: 43 was prepared and purified.

The GD2-SADA construct comprises a GD2 scFv (amino acid no: 1-252), a DOTA binding scFv (amino acids 275-533) and a SADA domain (amino acids 545-583). The construct comprises a disulfide bond between the VH and VL of the GD2 scFv, formed by the cysteines C97 and C179, and one disulfide bond between the VH and VL of the DOTA binding scFv, formed by the cysteines C369 and C513.

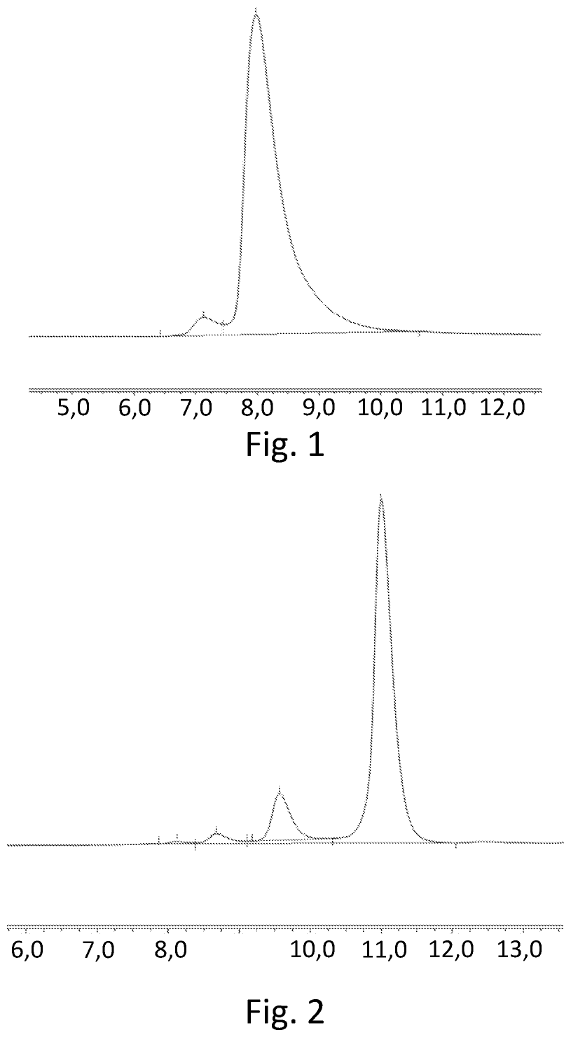

The purified construct was analyzed using SE-HPLC (see FIG. 1) and it was found that it was mainly in the tetrameric form, however, the peak appeared broad and a high molecular shoulder was observed suggesting that some inhomogeneity may be present in the peak.

In order to resolve the inhomogeneity, a truncated version of the GD2-SADA, called GD2-SADA minus P53 domain, was prepared where the molecule was truncated after amino acid G533, meaning that the SADA domain was lost.

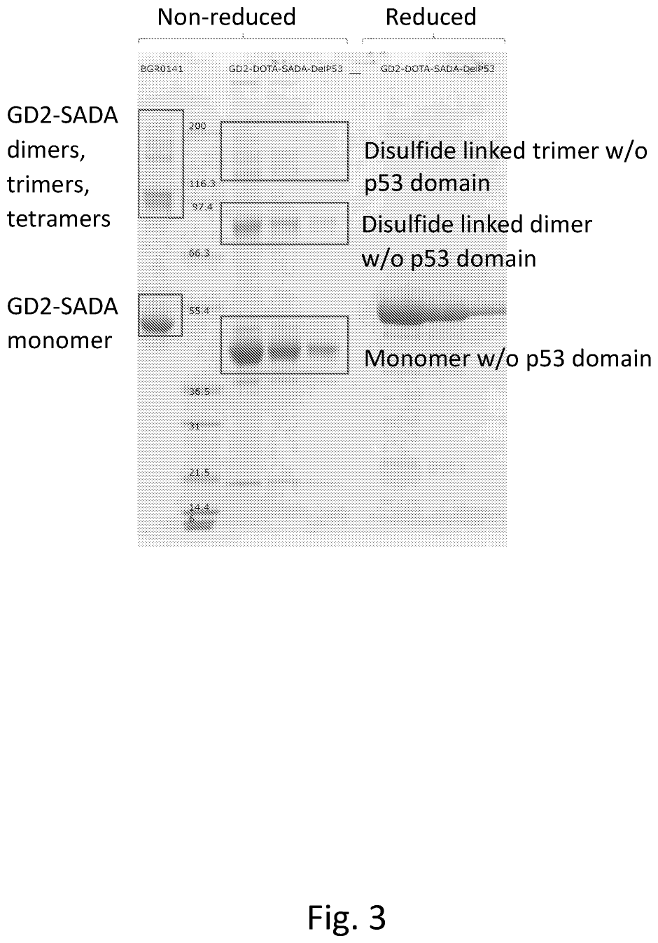

The truncated form was also analyzed by SE-HPLC, see FIG. 2. As expected, the truncated form lacked the ability to tetramerize due to the lack of the SADA domain, so the majority was found as monomers, but some dimer, trimer and tetramers could also be seen at the chromatogram (see FIG. 2).

The experiments showed that the GD2-SADA construct formed multimers, mainly dimers, and that the multimerization was not alone caused by the SADA domain.

### Example 2: SDS-PAGE Analysis of Multimers

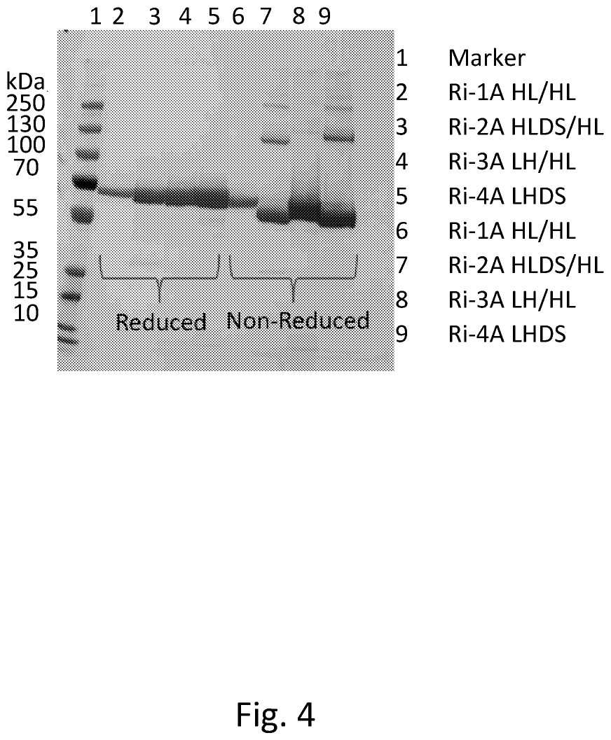

The GD2-SADA construct and the truncated form, prepared in Example 1, was further analyzed by SDS-PAGE chromatography, see FIG. 3.

GD2-SADA and truncated GD2-SADA were separated under non-reducing conditions and consistently showed the presence of multimers, in particular dimers and trimers. When the truncated form was analyzed under reducing conditions all forms collapsed into the monomeric form, confirming that the observed multimerization was caused by disulfide bonds.

### Example 3: CD20-SADA

In this example variants of bispecific antibodies capable of binding CD20 and DOTA was generated. The anti-CD20 site was varied in the order of VH and VL regions and with or without a disulfide bond connecting VH and VL. The DOTA binding site was the scFv disclosed in SEQ ID NO: 4 and the SADA domain was the domain disclosed in SEQ ID NO: 5.

The amino acid sequences of the VL sequence of the anti-CD20 scFv is disclosed in SEQ ID NO: 37, and for forming the anti-CD20 scFv without disulfide bond the cysteine in position 99 was substituted with a Glycine (G). The amino acid sequence of the VH sequence of the anti-CD20 scFv is disclosed in SEQ ID NO: 38, and for forming the anti-CD20 scFv without disulfide bond the cysteine in position 44 was substituted with a Glycine (G).

The sequence of the construct Ri-3A is disclosed in SEQ ID NO: 39.

Following constructs were generated

| | Anti-CD20 | Anti-DOTA | SADA |
|---|---|---|---|
| Ri-1A | VH-VL orientation, | VH-VL orientation, | P53 domain |
| | no disulfide | no disulfide | (SEQ ID |
| | between VH and VL | between VH and VL | NO: 5) |
| Ri-2A | VH-VL orientation, | VH-VL orientation, | P53 domain |
| | one disulfide | no disulfide | (SEQ ID |
| | between VH and VL | between VH and VL | NO: 5) |
| Ri-3A | VL-VH orientation, | VH-VL orientation, | P53 domain |
| | no disulfide | no disulfide | (SEQ ID |
| | between VH and VL | between VH and VL | NO: 5) |
| Ri-4A | VH-VL orientation, | VH-VL orientation, | P53 domain |
| | one disulfide | no disulfide | (SEQ ID |
| | between VH and VL | between VH and VL | NO: 5) |

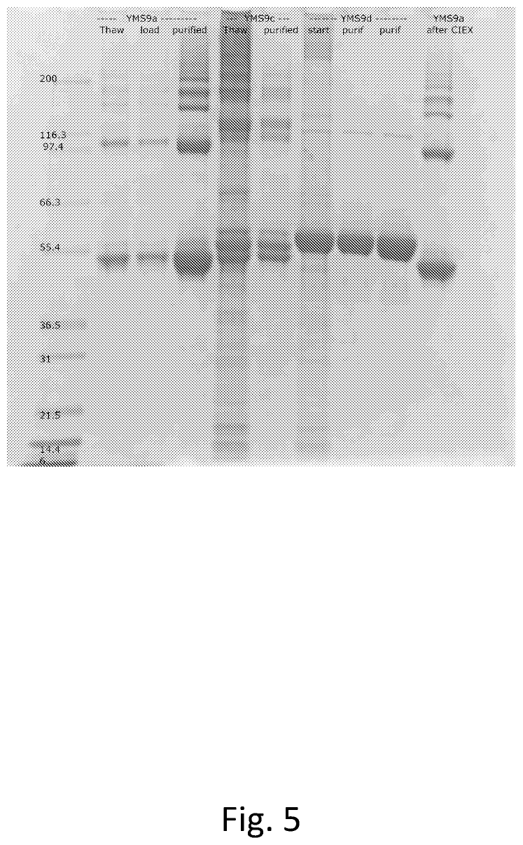

The four constructs were separated on SDS-PAGE under reducing and non-reducing conditions (See FIG. 4).

The figure shows that under non-reducing conditions, the constructs comprising a disulfide bond between VH and VH (Ri-2A and Ri-4A) formed high molecular weight multimers, and that the multimers content was strongly reduced or even absent in the constructs without a disulfide bond between VH and VH (Ri-1A and Ri-3A).

Under reducing conditions all four constructs collapsed into the monomeric form.

### Example 4: CD38-SADA

In this example variants of bispecific antibodies capable of binding CD38 and DOTA was generated. The DOTA binding site was based on the scFv disclosed in SEQ ID NO: 3 and is the scFv disclosed in SEQ ID NO: 4. The DOTA binding site comprising one disulfide bond between the VH and VL containing cysteines in positions 111 and 194. The SADA domain was the domain disclosed in SEQ ID NO: 5.

The amino acid sequence of the VL sequence of the anti-CD38 scFv is disclosed in SEQ ID NO: 28, and for forming the anti-CD38 scFv without disulfide bond the cysteine in position 100 was substituted with a Glutamine (Q). The amino acid sequence of the VH sequence is disclosed in SEQ ID NO: 29, and for forming the anti-CD38 scFv without disulfide bond the cysteine in position 44 was substituted with a Glycine (G).

Following constructs were generated

| | antiCD38 scFv | Anti-DOTA | SADA |
|---|---|---|---|
| YMS9a | Containing one VH- | Containing one VH- | P53 domain |
| | VL disulfide bond | VL disulfide bond | (SEQ ID NO: 5) |
| YMS9c | Containing one VH- | Without VH-VL | P53 domain |
| | VL disulfide bond | disulfide bond | (SEQ ID NO: 5) |
| YMS9d | Without VH-VL | Without VH-VL | P53 domain |
| | disulfide bond | disulfide bond | (SEQ ID NO: 5) |

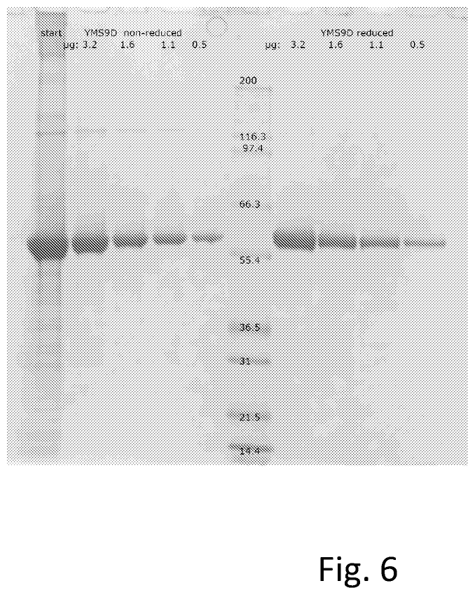

The constructs were analyzed on non-reducing SDS-PAGE, See FIG. 5.

The results show that YMS9a and YMS9c contained significant amounts of multimers, whereas the amount of multimers were significantly diminished or absent in YMS9d.

The results also showed that YMS9a and YMS9c gave rise to some heterogeneity in the monomer band. The heterogeneity disappeared under reducing conditions.

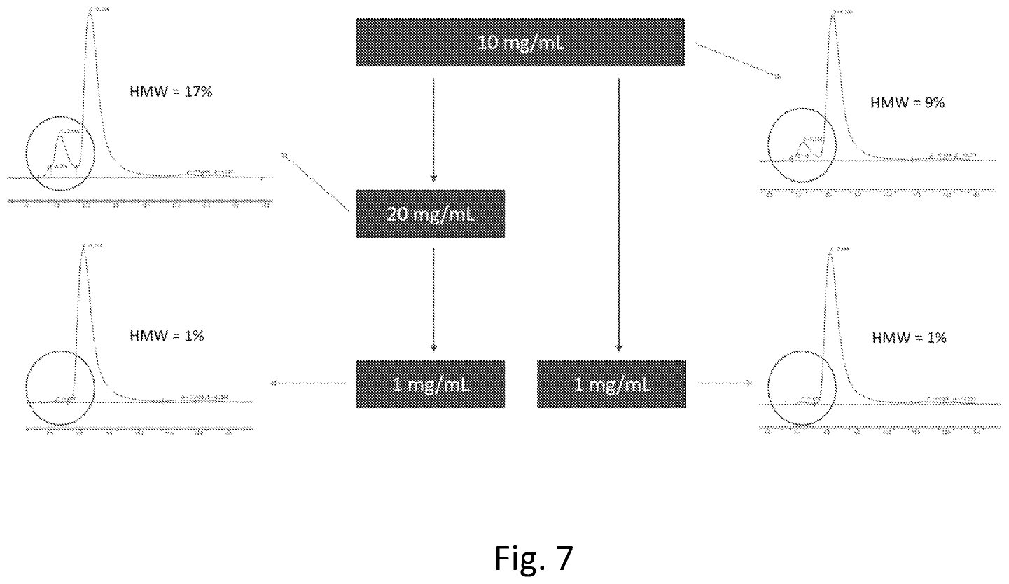

The YMS9d product was analysed further by loading various amounts, 3.2 μg, 1.6 μg, 1.1 μg and 0.5 μg on SDS-PAGE gel under non-reducing and reducing conditions. The results, shown in FIG. 6, showed that the protein was eluted as a single band under both reducing and non-reducing conditions, and only in the lanes with high protein load could a few additional faint bands be seen under non-reducing conditions.

### Example 5: CD38-SADA In Vitro Potency by SPR

In this example, the binding properties of a SADA construct of the invention were tested by SPR analysis.

The YMS9a (with a disulfide bond between the VL and VH of the DOTA binding site and a disulfide bond between the VL and VH of the CD38 binding site) and YMS9d (without disulfide bonds between VL and VH), prepared according to example 4, were analysed by SPR analysis both for binding to DOTA and for binding to CD38.

The results showed no significant difference for in vitro binding efficacy between YMS9a and YMS9d.

### Example 6. HMW Forms of CD38-SADA without Inter-Chain DS Bonds

The compound YMS9d, as prepared in example 4, and a solution of 10 mg/ml was prepared. After the solution was prepared, it was allowed to equilibrate for 3 hours at room temperature. The solution was analysed by SE-HPLC.

A sample of the stock solution was upconcentrated to 20 mg/ml. After the solution was prepared, it was allowed to equilibrate for 3 hours at room temperature. The solution was analysed by SE-HPLC.

The 10 mg/ml solution and the 20 mg/ml solutions were each diluted to 1 mg/ml. After the solution was prepared, it was allowed to equilibrate for 3 hours at room temperature. The solutions were analysed by SE-HPLC.

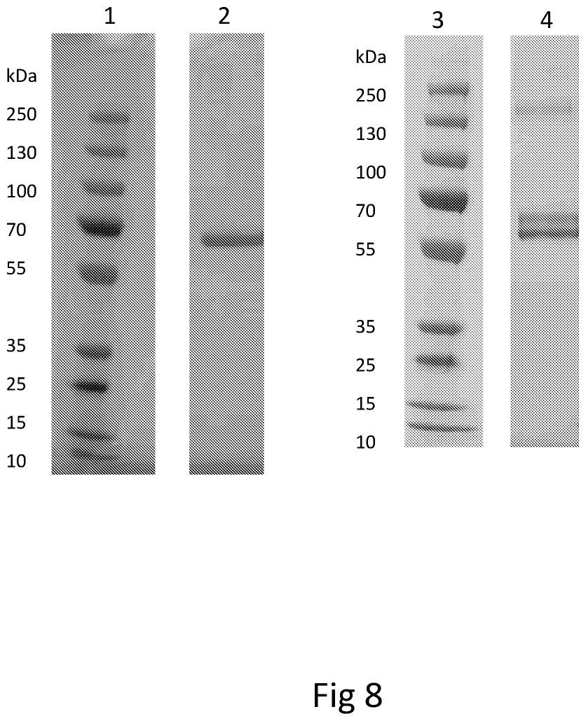

The results are shown in FIG. 7, and showed that the compound formed high molecular weight forms (the peaks encircled in FIG. 7) at high concentrations, and that practically all of these high molecular forms disassembled into tetramers upon dilution.

### Example 7. CD38 and Lu-DOTA Binding by SPR

The binding properties of samples diluted from 10 mg/ml and 20 mg/ml solutions as described in example 6, were analysed by SPR. Results are shown in the tables below:

Binding to CD38

| Sample | Ka (1/Ms) | Kd (1/s) | KD (nM) |
|---|---|---|---|
| Diluted from 10 mg/ml | 1.4 × 10⁵ | 1.1 × 10⁻³ | 7.5 |
| Diluted from 20 mg/ml | 1.3 × 10⁵ | 1.1 × 10⁻³ | 8.1 |

Binding to Lu-DOTA

| Sample | Ka (1/Ms) | Kd (1/s) | KD (nM) |
|---|---|---|---|
| Diluted from 10 mg/ml | 8.9 × 10⁴ | 2.1 × 10⁻³ | 2.4 |
| Diluted from 20 mg/ml | 8.6 × 10⁴ | 2.0 × 10⁻³ | 2.3 |

The results showed that the formation and subsequent disassembly of HMW forms did not significantly change the binding properties.

### Example 8: CD38-SADA In Vitro Binding to Daudi Cells

The compounds YMS9c, comprising one disulfide bond between the VH and VL chain in the CD38 scFv, and YMS9d, without any disulfide bonds between the VH and VL, were used in this example. The compounds were prepared as described in Example 4.

The compounds were labelled with ¹²⁵I and incubated with Daudi cells, comprising the CD38 antigen exposed on their surface. After the incubation the cells were rinsed and the radioactivity bound to the cells counted:

| | YMS9c (disulfide bond in | YMS9d (no disulfide bond |
|---|---|---|
| | CD38 scFv) | between VH and VL) |
| Bmax (cpm) | 462617 | 679547 |

The results showed that YMS9d, without VH-VL disulfides; had higher binding compared with YMS9c, with a disulfide bond on anti-CD38 site.

### Example 9: Biodistribution in Daudi Bearing Mice (In Vivo)

Daudi tumor bearing mice were given injections of 10 mg/kg of YMS9c, comprising one disulfide bond between the VH and VL chain in the CD38 scFv, and YMS9d, without any disulfide bonds between the VH and VL, as prepared in example 4.

48 h after administration of the CD38-SADA compounds, 5 MBq ¹⁷⁷Lu-DOTA/¹⁷⁷Lu-Bn-DOTA was administered to the mice.

2 hours and 24 hours after administration of radioactivity, the biodistribution was determined by euthanising and dissecting some mice (n=4) and counting the amount of radioactivity found in the selected tissues: Blood, tumor and kidney.

The tumor:blood ratios were calculated:

| | YMS9c (disulfide bond | | YMS9d (no disulfide bond | |
|---|---|---|---|---|
| | in CD38 scFv) | | in CD38 scFv) | |
| | 2 h | 24 h | 2 h | 24 h |
| Tumor:Blood | 0.389 | 8.345 | 1.989 | 62.218 |

The example showed higher tumor:blood uptake of the CD38-SADA Conjugate without disulfide bond between VL and VH compared with the conjugate with a disulfide bond between the VH and VL of the CD38 scFv.

### Example 10: RSV-SADA

In this example variants of bispecific antibodies capable of binding RSV and DOTA was generated. The DOTA binding site was the scFv disclosed in SEQ ID NO: 4 and the SADA domain was the domain disclosed in SEQ ID NO: 5.

Two versions of the RSV-SADA conjugate were generated, one version, PalDOT-SAD with a disulfide bond between the VL and VH of the RSV binding scFv, and one version, PA-3A without disulfide bonds between the VL and VH of the RSV binding scFv.

The sequence of the construct PA-3A is disclosed in SEQ ID NO: 62.

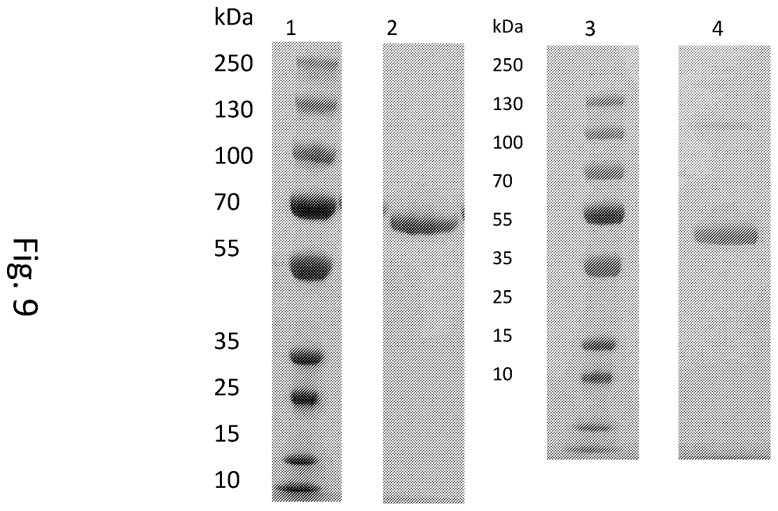

The two constructs were expressed and run on a non-reducing SDS-PAGE gel, as shown in FIG. 8, wherein lane 2 is PA-3A and lane 4 is PalDOT-SAD. The results showed that the construct without disulfide bonds between VH and VL ran as a single discrete band, whereas the version with one disulfide bond between VH and VL in the anti-RSV scFv showed the presence of several bands.

### Example 11: B7H3-SADA

In this example variants of bispecific antibodies capable of binding B7H3 and DOTA was generated. The DOTA binding site was the scFv disclosed in SEQ ID NO: 4 and the SADA domain was the domain disclosed in SEQ ID NO: 5.

Two versions of the B7H3-SADA conjugate were generated, one version, 3BH-4 with a disulfide bond between the VL and VH of the B7H3 binding scFv, and one version, 3BH-5 without disulfide bonds between the VL and VH of the B7H3 binding scFv.

The sequence of the construct 3BH-5 is disclosed in SEQ ID NO: 63.

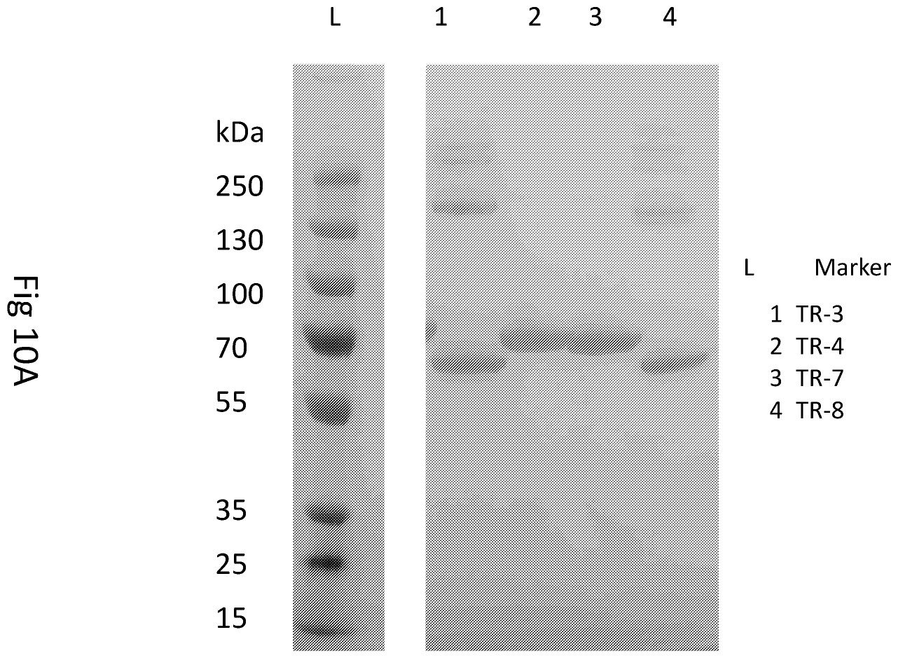

The two constructs were expressed and run on a non-reducing SDS-PAGE gel, as shown in FIG. 9, wherein lane 2 is 3BH-5 and lane 4 is 3BH-4. The results showed that the construct without disulfide bonds between VH and VL ran as a single discrete band, whereas the version with one disulfide bond between VH and VL in the anti-B7H3 scFv showed the presence of at least one higher molecular weight band.

### Example 12: HER2-SADA

In this example variants of bispecific antibodies capable of binding HER2 and DOTA were generated. The SADA domain was the domain disclosed in SEQ ID NO: 5.

Eight versions of the HER2-SADA conjugate were generated, one series (TR-series), using an anti-HER2 scFv derived from the clinical antibody, Trastuzumab, with four constructs:

| | | Anti- | Anti- |
|---|---|---|---|
| | | tumor | DOTA |
| Name | Design | DS | DS |
| TR-3 | Anti-HER2(VL-VH) × Anti-DOTA (VH-VL) | Yes | Yes |
| TR-4 | Anti-HER2(VL-VH) × Anti-DOTA (VH-VL) | No | No |
| TR-7 | Anti-HER2(VH-VL) × Anti-DOTA (VH-VL) | No | No |
| TR-8 | Anti-HER2(VH-VL) × Anti-DOTA (VH-VL) | No | Yes |

And one series (PE-series), using an antiHER2 scFv derived from the clinical antibody; Pertuzumab, with four constructs:

| | | Anti- | Anti- |
|---|---|---|---|
| | | tumor | DOTA |
| Name | Design | DS | DS |
| PE-1 | Anti-HER2(VH-VL) × Anti-DOTA (VH-VL) | No | No |
| PE-2 | Anti-HER2(VH-VL) × Anti-DOTA (VH-VL) | No | Yes |
| PE-3 | Anti-HER2(VL-VH) × Anti-DOTA (VH-VL) | No | No |
| PE-4 | Anti-HER2(VL-VH) × Anti-DOTA (VH-VL) | No | Yes |

The constructs of the TR series differ from the constructs of the PE series in that the VH and VL sequences of the anti-HER2 scFv site in the TR series are different from the VH and VL sequences of the anti-HER2 scFv site in the PE series.

The sequence of the construct TR-4 is disclosed in SEQ ID NO: 64.

The sequence of the construct TR-7 is disclosed in SEQ ID NO: 65.

The sequence of the construct PE-1 is disclosed in SEQ ID NO: 66.

The sequence of the construct PE-3 is disclosed in SEQ ID NO: 67.

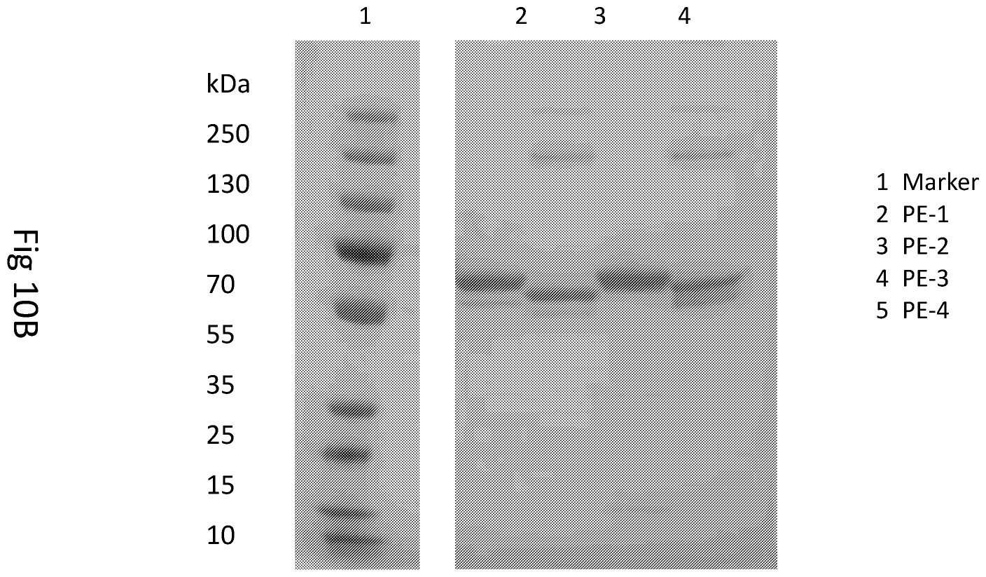

The constructs were expressed and run on a non-reducing SDS-PAGE gel. FIG. 10A shows the SDS-page gel of the TR series, and FIG. 10B shown the SDS-PAGE gel of the PE series. The results showed that the construct without disulfide bonds between VH and VL ran as a single discrete band, whereas the version with one disulfide bond between VH and VL in the HER2 scFv showed the presence of at least one high molecular weight band.

### Example 13: Anti-CD20-Anti-DOTAM-SADA

In this example variants of bispecific antibodies capable of binding CD20 and DOTAM was generated. The SADA domain was the domain disclosed in SEQ ID NO: 5.

Two versions of the Anti-CD20-Anti-DOTAM-SADA conjugate were generated:

| | | Anti- | Anti- |
|---|---|---|---|
| | | tumor | DOTA |
| Name | Design | DS | DS |
| Ri-12 | Anti-CD20(VL-VH) × Anti-DOTAM (VH-VL) | No | No |
| Ri-13 | Anti-CD20(VL-VH) × Anti-DOTAM (VL-VH) | No | No |

The sequence of the construct Ri-12 is disclosed in SEQ ID NO: 68.

The sequence of the construct Ri-13 is disclosed in SEQ ID NO: 69.

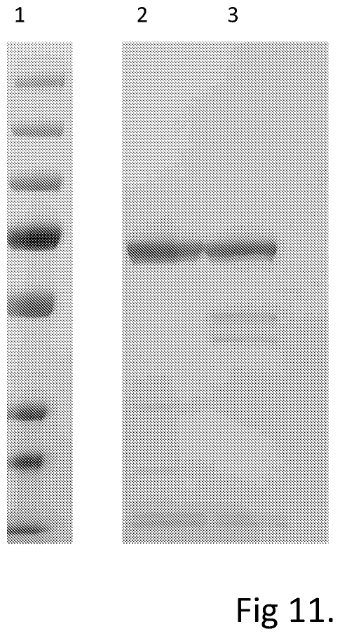

The two constructs were expressed and run on a non-reducing SDS-PAGE gel, as shown in FIG. 11, wherein lane 2 is Ri-12 and lane 3 is Ri-13.

### Sequences

SEQ ID NO. 1:
HVQLVESGGGLVQPGGSLRLSCAASGFSLTDYGVHWVRQAPGKGLEWLGVIWSGGGTAYNTALISRFTI
SRDNSKNTLYLQMNSLRAEDTAVYYCARRGSYPYNYFDAWGQGTLVTVSS
SEQ ID NO. 2:
QAVVTQEPSLTVSPGGTVTLTCGSSTGAVTASNYANWVQQKPGQAPRGLIGGHNNRPPGVPARFSGSL
LGGKAALTLLGAQPEDEAEYYCALWYSDHWVIGGGTKLTVLG
SEQ ID NO. 3:
HVKLQESGPGLVQPSQSLSLTCTVSGFSLTDYGVHWVRQSPGKGLEWLGVIWSGGGTAYNTALISRLNIY
RDNSKNQVFLEMNSLQAEDTAMYYCARRGSYPYNYFDAWGCGTTVTVSSGGGGSGGGGSGGGGSQA
VVIQESALTTPPGETVTLTCGSSTGAVTASNYANWVQEKPDHCFTGLIGGHNNRPPGVPARFSGSLIGDK
AALTIAGTQTEDEAIYFCALWYSDHWVIGGGTRLTVLG
SEQ ID NO. 4:
HVQLVESGGGLVQPGGSLRLSCAASGFSLTDYGVHWVRQAPGKGLEWLGVIWSGGGTAYNTALISRFTI
SRDNSKNTLYLQMNSLRAEDTAVYYCARRGSYPYNYFDAWGQGTLVTVSSGGGGSGGGGSGGGGSGG
GGSGGGGSGGGGSQAVVTQEPSLTVSPGGTVTLTCGSSTGAVTASNYANWVQQKPGQAPRGLIGGHN
NRPPGVPARFSGSLLGGKAALTLLGAQPEDEAEYYCALWYSDHWVIGGGTKLTVLG
SEQ ID NO. 5:
KPLDGEYFTLQIRGRERFEMFRELNEALELKDAQAGKEP
SEQ ID NO. 6:
RSPDDELLYLPVRGRETYEMLLKIKESLELMQYLPQHTIETYRQQQQQQHQHLLQK
SEQ ID NO. 7:
RHGDEDTYYLQVRGRENFEILMKLKESLELMELVPQPLVDSYRQQQQLLQRP
SEQ ID NO. 8:
QAIKKELTQIKQKVDSLLENLEKIEKE
SEQ ID NO. 9:
STRRILGLAIESQDAGIKTITMLDEQKEQLNRIEEGLDQINKDMRETEKTLTEL
SEQ ID NO. 10:
MCGAPSATQPATAETQHIADQVRSQLEEKENKKFPVFKAVSFKSQVVAGTNYFIKVHVGDEDFVHLRVF
QSLPHENKPLTLSNYQTNKAKHDELTYF
SEQ ID NO. 11:
DEISMMGRVVKVEKQVQSIEHKLDLLLGFY
SEQ ID NO. 12:
TVAEAKRQAAEDALAVINQQEDSSESCWNCGRKASETCSGCNTARYCGSFCQHKDWEKHH
SEQ ID NO. 13: KASQSVSNDVT
SEQ ID NO. 14: SASNRYS
SEQ ID NO. 15: QQDYSS
SEQ ID NO. 16: NYGVH
SEQ ID NO. 17: VIWAGGITNYNSAFMS
SEQ ID NO. 18: RGGHYGYALDY
SEQ ID NO. 19:
EIVMTQTPATLSVSAGERVTITCKASQSVSNDVTWYQQKPGQAPRLLIYSASNRYSGVPARFSGSGYGTE
FTFTISSVQSEDFAVYFCQQDYSSFGGGTKLEIKR
SEQ ID NO. 20:
QVQLVESGPGVVQPGRSLRISCAVSGFSVTNYGVHWVRQPPGKGLEWLGVIWAGGITNYNSAFMSRLT
ISKDNSKNTVYLQMNSLRAEDTAMYYCASRGGHYGYALDYWGQGTLVTVSS
SEQ ID NO. 21:
EIVMTQTPATLSVSAGERVTITCKASQSVSNDVTWYQQKPGQAPRLLIYSASNRYSGVPARFSGSGYGTE
FTFTISSVQSEDFAVYFCQQDYSSFGQGTKLEIKRGGGGSGGGGSGGGGSGGGGSGGGGSGGGGSQVQ
LVESGPGVVQPGRSLRISCAVSGFSVTNYGVHWVRQPPGKGLEWLGVIWAGGITNYNSAFMSRLTISKD
NSKNTVYLQMNSLRAEDTAMYYCASRGGHYGYALDYWGQGTLVTVSS
SEQ ID NO. 22: EDIYNR
SEQ ID NO. 23: GAT
SEQ ID NO. 24: QQYWSNPYT
SEQ ID NO. 25: GFSLTSYG
SEQ ID NO. 26: MWRGGST
SEQ ID NO. 27: AKSMITTGFVMDS
SEQ ID NO. 28:
DIQLTQSPSSLSASVGDRVTITCKASEDIYNRLTWYQQKPGKAPKLLISGATSLETGVPSRFSGSGSGKDYTF
TISSLQPEDFATYYCQQYWSNPYTFGQGTKLEIK
SEQ ID NO. 29:
QVQLQESGPGLVKPSETLSLTCTVSGFSLTSYGVHWVRQPPGKGLEWIGVMWRGGSTDYNAAFKSRVTI
SKDNSKNQVSLKLSSVTAADTAVYYCAKSMITTGFVMDSWGQGTLVTVSS
SEQ ID NO. 30:
QVQLQESGPGLVKPSETLSLTCTVSGFSLTSYGVHWVRQPPGKGLEWIGVMWRGGSTDYNAAFKSRVTI
SKDNSKNQVSLKLSSVTAADTAVYYCAKSMITTGFVMDSWGQGTLVTVSSGGGGSGGGGSGGGGSGG
GGSGGGGSGGGGSDIQLTQSPSSLSASVGDRVTITCKASEDIYNRLTWYQQKPGKAPKLLISGATSLETGV
PSRFSGSGSGKDYTFTISSLQPEDFATYYCQQYWSNPYTFGQGTKLEIK
SEQ ID NO. 31: SSVSY
SEQ ID NO. 32: ATS
SEQ ID NO. 33: QQWTSNPPT
SEQ ID NO. 34: GYTFTSYN
SEQ ID NO. 35: IYPGNGDT
SEQ ID NO. 36: ARSTYYGGDWYFNV
SEQ ID NO. 37:
QIVLSQSPAILSASPGEKVTMTCRASSSVSYIHWFQQKPGSSPKPWIYATSNLASGVPVRFSGSGSGTSYSL
TISRVEAEDAATYYCQQWTSNPPTFGGGTKLEIK
SEQ ID NO. 38:
QVQLQQPGAELVKPGASVKMSCKASGYTFTSYNMHWVKQTPGRGLEWIGAIYPGNGDTSYNQKFKGK
ATLTADKSSSTAYMQLSSLTSEDSAVYYCARSTYYGGDWYFNVWGAGTTVTVSA
SEQ ID NO. 39:
QIVLSQSPAILSASPGEKVTMTCRASSSVSYIHWFQQKPGSSPKPWIYATSNLASGVPVRFSGSGSGTSYSL
TISRVEAEDAATYYCQQWTSNPPTFGGGTKLEIKGGGGSGGGGSGGGGSGGGGSGGGGSGGGGSQV
QLQQPGAELVKPGASVKMSCKASGYTFTSYNMHWVKQTPGRGLEWIGAIYPGNGDTSYNQKFKGKAT
LTADKSSSTAYMQLSSLTSEDSAVYYCARSTYYGGDWYFNVWGAGTTVTVSA
SEQ ID NO. 40:
EIVMTQTPATLSVSAGERVTITCKASQSVSNDVTWYQQKPGQAPRLLIYSASNRYSGVPARFSGSGYGTE
FTFTISSVQSEDFAVYFCQQDYSSFGQGTKLEIKRGGGGSGGGGSGGGGSGGGGSGGGGSGGGGSQVQ
LVESGPGVVQPGRSLRISCAVSGFSVTNYGVHWVRQPPGKGLEWLGVIWAGGITNYNSAFMSRLTISKD
NSKNTVYLQMNSLRAEDTAMYYCASRGGHYGYALDYWGQGTLVTVSSGGGGSGGGGSGGGGSGGG
GSHVQLVESGGGLVQPGGSLRLSCAASGFSLTDYGVHWVRQAPGKGLEWLGVIWSGGGTAYNTALISR
FTISRDNSKNTLYLQMNSLRAEDTAVYYCARRGSYPYNYFDAWGQGTLVTVSSGGGGSGGGGSGGGGS
GGGGSGGGGSGGGGSQAVVTQEPSLTVSPGGTVTLTCGSSTGAVTASNYANWVQQKPGQAPRGLIGG
HNNRPPGVPARFSGSLLGGKAALTLLGAQPEDEAEYYCALWYSDHWVIGGGTKLTVLGTPLGDTTHTSG
KPLDGEYFTLQIRGRERFEMFRELNEALELKDAQAGKEPGGSGGA
SEQ ID NO. 41:
QVQLQESGPGLVKPSETLSLTCTVSGFSLTSYGVHWVRQPPGKGLEWIGVMWRGGSTDYNAAFKSRVTI
SKDNSKNQVSLKLSSVTAADTAVYYCAKSMITTGFVMDSWGQGTLVTVSSGGGGSGGGGSGGGGSGG
GGSGGGGSGGGGSDIQLTQSPSSLSASVGDRVTITCKASEDIYNRLTWYQQKPGKAPKLLISGATSLETGV
PSRFSGSGSGKDYTFTISSLQPEDFATYYCQQYWSNPYTFGQGTKLEIKGGGGSGGGGSGGGGSGGGGS
HVQLVESGGGLVQPGGSLRLSCAASGFSLTDYGVHWVRQAPGKGLEWLGVIWSGGGTAYNTALISRFTI
SRDNSKNTLYLQMNSLRAEDTAVYYCARRGSYPYNYFDAWGQGTLVTVSSGGGGSGGGGSGGGGSGG
GGSGGGGSGGGGSQAVVTQEPSLTVSPGGTVTLTCGSSTGAVTASNYANWVQQKPGQAPRGLIGGHN
NRPPGVPARFSGSLLGGKAALTLLGAQPEDEAEYYCALWYSDHWVIGGGTKLTVLGTPLGDTTHTSGKPL
DGEYFTLQIRGRERFEMFRELNEALELKDAQAGKEPGGSGGAP
SEQ ID NO. 42:
QIVLSQSPAILSASPGEKVTMTCRASSSVSYIHWFQQKPGSSPKPWIYATSNLASGVPVRFSGSGSGTSYSL
TISRVEAEDAATYYCQQWTSNPPTFGGGTKLEIKGGGGSGGGGSGGGGSGGGGSGGGGGGGGSQV
QLQQPGAELVKPGASVKMSCKASGYTFTSYNMHWVKQTPGRGLEWIGAIYPGNGDTSYNQKFKGKAT
LTADKSSSTAYMQLSSLTSEDSAVYYCARSTYYGGDWYFNVWGAGTTVTVSAGGGGSGGGGSGGGGS
GGGGSHVQLVESGGGLVQPGGSLRLSCAASGFSLTDYGVHWVRQAPGKGLEWLGVIWSGGGTAYNTA
LISRFTISRDNSKNTLYLQMNSLRAEDTAVYYCARRGSYPYNYFDAWGQGTLVTVSSGGGGSGGGGSGG
GGSGGGGSGGGGSGGGGSQAVVTQEPSLTVSPGGTVTLTCGSSTGAVTASNYANWVQQKPGQAPRG
LIGGHNNRPPGVPARFSGSLLGGKAALTLLGAQPEDEAEYYCALWYSDHWVIGGGTKLTVLGTPLGDTT
HTSGKPLDGEYFTLQIRGRERFEMFRELNEALELKDAQAGKEPGGSGGA
SEQ ID NO. 43:
EIVMTQTPATLSVSAGERVTITCKASQSVSNDVTWYQQKPGQAPRLLIYSASNRYSGVPARFSGSGYGTE
FTFTISSVQSEDFAVYFCQQDYSSFGCGTKLEIKRGGGGSGGGGSGGGGSGGGGSGGGGSGGGGSQVQ
LVESGPGVVQPGRSLRISCAVSGFSVTNYGVHWVRQPPGKCLEWLGVIWAGGITNYNSAFMSRLTISKD
NSKNTVYLQMNSLRAEDTAMYYCASRGGHYGYALDYWGQGTLVTVSSGGGGSGGGGSGGGGSGGG
GSHVQLVESGGGLVQPGGSLRLSCAASGFSLTDYGVHWVRQAPGKGLEWLGVIWSGGGTAYNTALISR
FTISRDNSKNTLYLQMNSLRAEDTAVYYCARRGSYPYNYFDAWGCGTLVTVSSGGGGSGGGGSGGGGS
GGGGSGGGGSGGGGSQAVVTQEPSLTVSPGGTVTLTCGSSTGAVTASNYANWVQQKPGQCPRGLIGG
HNNRPPGVPARFSGSLLGGKAALTLLGAQPEDEAEYYCALWYSDHWVIGGGTKLTVLGTPLGDTTHTSG
KPLDGEYFTLQIRGRERFEMFRELNEALELKDAQAGKEPGGSGGA
SEQ ID NO. 44: TGAVTASNY
SEQ ID NO. 45: GHN
SEQ ID NO. 46: ALWYSDHWV
SEQ ID NO. 47: GFSLTDYG
SEQ ID NO. 48: IWSGGGT
SEQ ID NO. 49: ARRGSYPYNYFDA
SEQ ID NO. 50: GFAFSTYD
SEQ ID NO. 51: ISSGGSYT
SEQ ID NO. 52: APTTVVPFAY
SEQ ID NO. 53: QNVRTV
SEQ ID NO. 54: LAS
SEQ ID NO. 55: LQHWSYPLT
SEQ ID NO. 56:
EVQLVESGGGLVKPGGSLRLSCAASGFAFSTYDMSWVRQAPGKGLEWVSTISSGGSYTYYADSVKGRFTI
SRDNAKNSLYLQMNSLRAEDTAVYYCAPTTVVPFAYWGQGTLVTVSA
SEQ ID NO. 57:
DIQMTQSPSSLSASVGDRVTITCKASQNVRTVVAWYQQKPGKAPKTLIYLASNRHTGVPSRFSGSGSGTE
FTLTISNLQPEDFATYYCLQHWSYPLTFGSGTKLEVKR
SEQ ID NO. 58:
EVQLVESGGGLVKPGGSLRLSCAASGFAFSTYDMSWVRQAPGKGLEWVSTISSGGSYTYYADSVKGRFTI
SRDNAKNSLYLQMNSLRAEDTAVYYCAPTTVVPFAYWGQGTLVTVSAGGGGSGGGGSGGGGSGGGGS
GGGGSGGGGSDIQMTQSPSSLSASVGDRVTITCKASQNVRTVVAWYQQKPGKAPKTLIYLASNRHTGV
PSRFSGSGSGTEFTLTISNLQPEDFATYYCLQHWSYPLTFGSGTKLEVKRGGGGSGGGGSGGGGSGGGG
SHVQLVESGGGLVQPGGSLRLSCAASGFSLTDYGVHWVRQAPGKGLEWLGVIWSGGGTAYNTALISRFT
ISRDNSKNTLYLQMNSLRAEDTAVYYCARRGSYPYNYFDAWGQGTLVTVSSGGGGSGGGGGGGGSG
GGGSGGGGSGGGGSQAVVTQEPSLTVSPGGTVTLTCGSSTGAVTASNYANWVQQKPGQAPRGLIGGH
NNRPPGVPARFSGSLLGGKAALTLLGAQPEDEAEYYCALWYSDHWVIGGGTKLTVLGTPLGDTTHTSGK
PLDGEYFTLQIRGRERFEMFRELNEALELKDAQAGKEPGGSGGAP
SEQ ID NO. 59:
EVQLVESGGGLVKPGGSLRLSCAASGFAFSTYDMSWVRQAPGKGLEWVSTISSGGSYTYYADSVKGRFTI
SRDNAKNSLYLQMNSLRAEDTAVYYCAPTTVVPFAYWGQGTLVTVSAGGGGSGGGGSGGGGSGGGGS
GGGGSGGGGSDIQMTQSPSSLSASVGDRVTITCKASQNVRTVVAWYQQKPGKAPKTLIYLASNRHTGV
PSRFSGSGSGTEFTLTISNLQPEDFATYYCLQHWSYPLTFGSGTKLEVKRGGGGSGGGGSGGGGSGGGG
SHVQLVESGGGLVQPGGSLRLSCAASGFSLTDYGVHWVRQAPGKGLEWLGVIWSGGGTAYNTALISRFT
ISRDNSKNTLYLQMNSLRAEDTAVYYCARRGSYPYNYFDAWGCGTLVTVSSGGGGGGGGSGGGGSGG
GGSGGGGSGGGGSQAVVTQEPSLTVSPGGTVTLTCGSSTGAVTASNYANWVQQKPGQCPRGLIGGHN
NRPPGVPARFSGSLLGGKAALTLLGAQPEDEAEYYCALWYSDHWVIGGGTKLTVLGTPLGDTTHTSGKPL
DGEYFTLQIRGRERFEMFRELNEALELKDAQAGKEPGGSGGAP
SEQ ID NO. 60:
EVQLVESGGGLVKPGGSLRLSCAASGFAFSTYDMSWVRQAPGKCLEWVSTISSGGSYTYYADSVKGRFTI
SRDNAKNSLYLQMNSLRAEDTAVYYCAPTTVVPFAYWGQGTLVTVSAGGGGSGGGGSGGGGSGGGGS
GGGGSGGGGSDIQMTQSPSSLSASVGDRVTITCKASQNVRTVVAWYQQKPGKAPKTLIYLASNRHTGV
PSRFSGSGSGTEFTLTISNLQPEDFATYYCLQHWSYPLTFGCGTKLEVKRGGGGSGGGGSGGGGSGGGG
SHVQLVESGGGLVQPGGSLRLSCAASGFSLTDYGVHWVRQAPGKGLEWLGVIWSGGGTAYNTALISRFT
ISRDNSKNTLYLQMNSLRAEDTAVYYCARRGSYPYNYFDAWGCGTLVTVSSGGGGSGGGGSGGGGSGG
GGSGGGGSGGGGSQAVVTQEPSLTVSPGGTVTLTCGSSTGAVTASNYANWVQQKPGQCPRGLIGGHN
NRPPGVPARFSGSLLGGKAALTLLGAQPEDEAEYYCALWYSDHWVIGGGTKLTVLGTPLGDTTHTSGKPL
DGEYFTLQIRGRERFEMFRELNEALELKDAQAGKEPGGSGGAP
SEQ ID NO. 61:
EVQLVESGGGLVKPGGSLRLSCAASGFAFSTYDMSWVRQAPGKGLEWVSTISSGGSYTYYADSVKGRFTI
SRDNAKNSLYLQMNSLRAEDTAVYYCAPTTVVPFAYWGQGTLVTVSAGGGGSGGGGSGGGGSGGGGS
GGGGSGGGGSDIQMTQSPSSLSASVGDRVTITCKASQNVRTVVAWYQQKPGKAPKTLIYLASNRHTGV
PSRFSGSGSGTEFTLTISNLQPEDFATYYCLQHWSYPLTFGSGTKLEVKR
SEQ ID NO. 62:
QVTLRESGPALVKPTQTLTLTCTFSGFSLSTSGMSVGWIRQPPGKALEWLADIWWDDKKDYNPSLKSRLT
ISKDTSKNQVVLKVTNMDPADTATYYCARSMITNWYFDVWGAGTTVTVSSGGGGSGGGGSGGGGSG
GGGSGGGGSGGGGSDIQMTQSPSTLSASVGDRVTITCKSQLSVGYMHWYQQKPGKAPKLLIYDTSKLAS
GVPSRFSGSGSGTEFTLTISSLQPDDFATYYCFQGSGYPFTFGGGTKLEIKGGGGSGGGGSGGGGSGGGG
SHVQLVESGGGLVQPGGSLRLSCAASGFSLTDYGVHWVRQAPGKGLEWLGVIWSGGGTAYNTALISRFT
ISRDNSKNTLYLQMNSLRAEDTAVYYCARRGSYPYNYFDAWGQGTLVTVSSGGGGGGGGSGGGGSG
GGGSGGGGSGGGGSQAVVTQEPSLTVSPGGTVTLTCGSSTGAVTASNYANWVQQKPGQAPRGLIGGH
NNRPPGVPARFSGSLLGGKAALTLLGAQPEDEAEYYCALWYSDHWVIGGGTKLTVLGTPLGDTTHTSGK
PLDGEYFTLQIRGRERFEMFRELNEALELKDAQAGKEPGGSGGA
SEQ ID NO. 63:
QVQLVQSGAEVKKPGASVKVSCKASGYTFTNYDINWVRQATGQGLEWMGWIFPGDGSTQYNEKFQG
RVTMTTNTSISTAYMELSSLRSEDTAVYYCARQTTATWFAYWGQGTLVTVSSGGGGSGGGGSGGGGSG
GGGSGGGGSGGGGSEIVMTQSPATLSVTPKEKVTITCRASQSISDYLHWYQQKPDQSPKLLIKYASQSISG
VPSRFSGSGSGSDFTLTINSLEAEDAATYYCQNGHSFPLTFGQGTKLEIKGGGGGGGGSGGGGSGGGGS
HVQLVESGGGLVQPGGSLRLSCAASGFSLTDYGVHWVRQAPGKGLEWLGVIWSGGGTAYNTALISRFTI
SRDNSKNTLYLQMNSLRAEDTAVYYCARRGSYPYNYFDAWGQGTLVTVSSGGGGSGGGGSGGGGSGG
GGSGGGGSGGGGSQAVVTQEPSLTVSPGGTVTLTCGSSTGAVTASNYANWVQQKPGQAPRGLIGGHN
NRPPGVPARFSGSLLGGKAALTLLGAQPEDEAEYYCALWYSDHWVIGGGTKLTVLGTPLGDTTHTSGKPL
DGEYFTLQIRGRERFEMFRELNEALELKDAQAGKEPGGSGGAP
SEQ ID NO. 64:
DIQMTQSPSSLSASVGDRVTITCRASQDVNTAVAWYQQKPGKAPKLLIYSASFLYSGVPSRFSGSRSGTDF
TLTISSLQPEDFATYYCQQHYTTPPTFGQGTKVEIKRGGGGSGGGGSGGGGSGGGGSGGGGGGGGSE
VQLVESGGGLVQPGGSLRLSCAASGFNIKDTYIHWVRQAPGKGLEWVARIYPTNGYTRYADSVKGRFTIS
ADTSKNTAYLQMNSLRAEDTAVYYCSRWGGDGFYAMDYWGQGTLVTVSSGGGGSGGGGSGGGGSG
GGGSHVQLVESGGGLVQPGGSLRLSCAASGFSLTDYGVHWVRQAPGKGLEWLGVIWSGGGTAYNTALI
SRFTISRDNSKNTLYLQMNSLRAEDTAVYYCARRGSYPYNYFDAWGQGTLVTVSSGGGGSGGGGSGGG
GSGGGGSGGGGGGGGSQAVVTQEPSLTVSPGGTVTLTCGSSTGAVTASNYANWVQQKPGQAPRGLI
GGHNNRPPGVPARFSGSLLGGKAALTLLGAQPEDEAEYYCALWYSDHWVIGGGTKLTVLGTPLGDTTHT
SGKPLDGEYFTLQIRGRERFEMFRELNEALELKDAQAGKEPGGSGGAP
SEQ ID NO. 65:
EVQLVESGGGLVQPGGSLRLSCAASGFNIKDTYIHWVRQAPGKGLEWVARIYPTNGYTRYADSVKGRFTI
SADTSKNTAYLQMNSLRAEDTAVYYCSRWGGDGFYAMDYWGQGTLVTVSSGGGGSGGGGSGGGGS
GGGGSGGGGSGGGGSDIQMTQSPSSLSASVGDRVTITCRASQDVNTAVAWYQQKPGKAPKLLIYSASFL
YSGVPSRFSGSRSGTDFTLTISSLQPEDFATYYCQQHYTTPPTFGQGTKVEIKRGGGGGGGGSGGGGSG
GGGSHVQLVESGGGLVQPGGSLRLSCAASGFSLTDYGVHWVRQAPGKGLEWLGVIWSGGGTAYNTALI
SRFTISRDNSKNTLYLQMNSLRAEDTAVYYCARRGSYPYNYFDAWGQGTLVTVSSGGGGSGGGGSGGG
GSGGGGSGGGGSGGGGSQAVVTQEPSLTVSPGGTVTLTCGSSTGAVTASNYANWVQQKPGQAPRGLI
GGHNNRPPGVPARFSGSLLGGKAALTLLGAQPEDEAEYYCALWYSDHWVIGGGTKLTVLGTPLGDTTHT
SGKPLDGEYFTLQIRGRERFEMFRELNEALELKDAQAGKEPGGSGGAP
SEQ ID NO. 66:
EVQLVESGGGLVQPGGSLRLSCAASGFTFTDYTMDWVRQAPGKGLEWVADVNPNSGGSIYNQRFKGR
FTLSVDRSKNTLYLQMNSLRAEDTAVYYCARNLGPSFYFDYWGQGTLVTVSSGGGGSGGGGSGGGGSG
GGGSGGGGSGGGGSDIQMTQSPSSLSASVGDRVTITCKASQDVSIGVAWYQQKPGKAPKLLIYSASYRY
TGVPSRFSGSGSGTDFTLTISSLQPEDFATYYCQQYYIYPYTFGQGTKVEIKGGGGSGGGGSGGGGSGGG
GSHVQLVESGGGLVQPGGSLRLSCAASGFSLTDYGVHWVRQAPGKGLEWLGVIWSGGGTAYNTALISR
FTISRDNSKNTLYLQMNSLRAEDTAVYYCARRGSYPYNYFDAWGQGTLVTVSSGGGGSGGGGSGGGGS
GGGGSGGGGSGGGGSQAVVTQEPSLTVSPGGTVTLTCGSSTGAVTASNYANWVQQKPGQAPRGLIGG
HNNRPPGVPARFSGSLLGGKAALTLLGAQPEDEAEYYCALWYSDHWVIGGGTKLTVLGTPLGDTTHTSG
KPLDGEYFTLQIRGRERFEMFRELNEALELKDAQAGKEPGGSGGAP
SEQ ID NO. 67:
DIQMTQSPSSLSASVGDRVTITCKASQDVSIGVAWYQQKPGKAPKLLIYSASYRYTGVPSRFSGSGSGTDF
TLTISSLQPEDFATYYCQQYYIYPYTFGQGTKVEIKGGGGSGGGGSGGGGSGGGGSGGGGSGGGGSEVQ
LVESGGGLVQPGGSLRLSCAASGFTFTDYTMDWVRQAPGKGLEWVADVNPNSGGSIYNQRFKGRFTLS
VDRSKNTLYLQMNSLRAEDTAVYYCARNLGPSFYFDYWGQGTLVTVSSGGGGSGGGGSGGGGGGGG
SHVQLVESGGGLVQPGGSLRLSCAASGFSLTDYGVHWVRQAPGKGLEWLGVIWSGGGTAYNTALISRFT
ISRDNSKNTLYLQMNSLRAEDTAVYYCARRGSYPYNYFDAWGQGTLVTVSSGGGGSGGGGGGGGSG
GGGSGGGGSGGGGSQAVVTQEPSLTVSPGGTVTLTCGSSTGAVTASNYANWVQQKPGQAPRGLIGGH
NNRPPGVPARFSGSLLGGKAALTLLGAQPEDEAEYYCALWYSDHWVIGGGTKLTVLGTPLGDTTHTSGK
PLDGEYFTLQIRGRERFEMFRELNEALELKDAQAGKEPGGSGGAP
SEQ ID NO. 68:
QIVLSQSPAILSASPGEKVTMTCRASSSVSYIHWFQQKPGSSPKPWIYATSNLASGVPVRFSGSGSGTSYSL
TISRVEAEDAATYYCQQWTSNPPTFGGGTKLEIKGGGGSGGGGSGGGGSGGGGSGGGGSGGGGSQV
QLQQPGAELVKPGASVKMSCKASGYTFTSYNMHWVKQTPGRGLEWIGAIYPGNGDTSYNQKFKGKAT
LTADKSSSTAYMQLSSLTSEDSAVYYCARSTYYGGDWYFNVWGAGTTVTVSAGGGGSGGGGSGGGGS
GGGGSVTLKESGPVLVKPTETLTLTCTVSGFSLSTYSMSWIRQPPGKALEWLGFIGSRGDTYYASWAKGR
LTISKDTSKSQVVLTMTNMDPVDTATYYCARERDPYGGGAYPPHLWGRGTLVTVSSGGGGSGGGGSGG
GGSGGGGSGGGGSGGGGSSIQMTQSPSSLSASVGDRVTITCQSSHSVYSDNDLAWYQQKPGKAPKLLIY
QASKLASGVPSRFSGSGSGTDFTLTISSLQPEDFATYYCLGGYDDESDTYGFGGGTKVEIKTPLGDTTHTSG
KPLDGEYFTLQIRGRERFEMFRELNEALELKDAQAGKEPGGSGGA
SEQ ID NO. 69:
QIVLSQSPAILSASPGEKVTMTCRASSSVSYIHWFQQKPGSSPKPWIYATSNLASGVPVRFSGSGSGTSYSL
TISRVEAEDAATYYCQQWTSNPPTFGGGTKLEIKGGGGSGGGGSGGGGSGGGGSGGGGSGGGGSQV
QLQQPGAELVKPGASVKMSCKASGYTFTSYNMHWVKQTPGRGLEWIGAIYPGNGDTSYNQKFKGKAT
LTADKSSSTAYMQLSSLTSEDSAVYYCARSTYYGGDWYFNVWGAGTTVTVSAGGGGSGGGGSGGGGS
GGGGSSIQMTQSPSSLSASVGDRVTITCQSSHSVYSDNDLAWYQQKPGKAPKLLIYQASKLASGVPSRFS
GSGSGTDFTLTISSLQPEDFATYYCLGGYDDESDTYGFGGGTKVEIKGGGGSGGGGSGGGGSGGGGSGG
GGSGGGGSVTLKESGPVLVKPTETLTLTCTVSGFSLSTYSMSWIRQPPGKALEWLGFIGSRGDTYYASWA
KGRLTISKDTSKSQVVLTMTNMDPVDTATYYCARERDPYGGGAYPPHLWGRGTLVTVSSTPLGDTTHTS
GKPLDGEYFTLQIRGRERFEMFRELNEALELKDAQAGKEPGGSGGA

1. A bispecific antibody comprising a first scFv domain capable of binding a chelator or a chelator binding a metal ion, a second scFv domain capable of binding a tumor antigen, and a SADA domain, wherein the VH and VL domains in the first scFv and/or the second scFv domain is/are not connected by a disulfide bond

2. The bispecific antibody of claim 1, wherein the first scFv domain does not comprise a disulfide bond connecting the VH and the VL and the second scFv domain does not comprise a disulfide bond connecting the VH and the VL.

3. The bispecific antibody of claim 1 or 2, further comprising one or more linker sequences.

4. The bispecific antibody according to any of the preceding claims, wherein the first scFv domain capable of binding a chelator or a chelator binding a metal ion, is selected among scFvs capable of binding DOTA, a derivative of DOTA, DOTAM or any of these binding a metal ion.

5. The bispecific antibody of claim 4, wherein the first scFv is capable of binding DOTA-metal, and comprises 6 CDR sequences each consisting of the sequences of SEQ ID NO: 44-49 or sequences that differs by 1 or 2 substitutions from the sequences of SEQ ID NO: 44-49.

6. The bispecific antibody of claim 5, comprising
a. a VL sequence with the sequence of SEQ ID NO: 1 or a sequence having at least 90% sequence identity, e.g. at least 95% sequence identity, e.g. at least 96% sequence identity, e.g. at least 97% sequence identity, e.g. at least 98% sequence identity or at least 99% sequence identity to SEQ ID NO: 1, wherein the amino acid in position 111 is not a cysteine; and
b. a VH sequence with the sequence of SEQ ID NO: 2 or a sequence having at least 90% sequence identity, e.g. at least 95% sequence identity, e.g. at least 96% sequence identity, e.g. at least 97% sequence identity, e.g. at least 98% sequence identity or at least 99% sequence identity to SEQ ID NO: 2, wherein the amino acid in position 45 is not a cysteine.

7. The bispecific antibody of claim 5 or 6, wherein the first scFv comprises or consists of the sequence of SEQ ID NO: 4, or comprising or consisting of a sequence having at least 90% sequence identity, e.g. at least 95% sequence identity, e.g. at least 96% sequence identity, e.g. at least 97% sequence identity, e.g. at least 98% sequence identity or at least 99% sequence identity to SEQ ID NO: 4.

8. The bispecific antibody of claim 4, wherein the first scFv is capable of binding DOTAM, and comprises 6 CDR sequences consisting of amino acids 302-310, 327-333, 372-387, 455-462, 480-482 and 519-530 of SEQ ID NO: 68 or sequences that differ from these sequences by 1 or 2 substitutions.

9. The bispecific antibody of claim 8, comprising
a. a VL sequence with the sequence of amino acids 429-540 of SEQ ID NO: 68 or a sequence having at least 90% sequence identity, e.g., at least 95% sequence identity, e.g., at least 96% sequence identity, e.g., at least 97% sequence identity, e.g., at least 98% sequence identity or at least 99% sequence identity to amino acids 429-540 of SEQ ID NO: 68; and
b. a VH sequence with the sequence of amino acids 278-398 of SEQ ID NO: 68, or a sequence having at least 90% sequence identity, e.g., at least 95% sequence identity, e.g., at least 96% sequence identity, e.g., at least 97% sequence identity, e.g., at least 98% sequence identity or at least 99% sequence identity to amino acids 278-398 of SEQ ID NO: 68.

10. The bispecific antibody of claim 8 or 9, wherein the first scFv comprises or consists of the sequence of amino acids 278-540 of SEQ ID NO: 68 or amino acids 278-540 of SEQ ID NO: 69, or comprising or consisting of a sequence having at least 90% sequence identity, e.g., at least 95% sequence identity, e.g., at least 96% sequence identity, e.g., at least 97% sequence identity, e.g., at least 98% sequence identity or at least 99% sequence identity to amino acids 278-540 of SEQ ID NO: 68 or amino acids 278-540 of SEQ ID NO: 69.

11. The bispecific antibody according to any of the preceding claims, wherein the tumor antigen is selected among: HER2, B7-H3, CA6, CD138, CD20, CD19, CD22, CD27L, CD30, CD33, CD37, CD38, CD47, CD56, CD66e, CD70, CD74, CD79b, EGFR, EGFRvIII, FRα, GCC, GPNMB, Mesothelin, MUC16, NaPi2b, Nectin 4, PSMA, STEAP1, Trop-2, 5T4, AGS-16, alpha v beta6, CA19.9, CAIX, CD138, CD174, CD180, CD227, CD326, CD79a, CEACAM5, CRIPTO, DLL3, DS6, Endothelin B receptor, FAP, GD2, Mesothelin, PMEL 17, SLC44A4, TENB2, TIM-1, CD98, Endosialin/CD248/TEM1, Fibronectin Extra-domain B, LIV-1, Mucin 1, p-cadherin, peritosin, Fyn, SLTRK6, Tenascin c, VEGFR2, and PRLR.

12. The bispecific antibody according to claim 11, wherein the tumor antigen is selected among: GD2, CD38, CD20, B7-H3, GPA33, RSV or HER2.

13. The bispecific antibody of claim 12, wherein the second scFv is capable of binding GD2, and comprises 6 CDR sequences each consisting of the sequences of SEQ ID NO: 13-18 or sequences that differs by 1 or 2 substitutions from the sequences of SEQ ID NO: 13-18.

14. The bispecific antibody of claim 13, comprising
a. a VL sequence with the sequence of SEQ ID NO: 19 or a sequence having at least 90% sequence identity, e.g. at least 95% sequence identity, e.g. at least 96% sequence identity, e.g. at least 97% sequence identity, e.g. at least 98% sequence identity or at least 99% sequence identity to SEQ ID NO: 19, wherein the amino acid in position 97 is not a cysteine; and
b. a VH sequence with the sequence of SEQ ID NO: 20 or a sequence having at least 90% sequence identity, e.g. at least 95% sequence identity, e.g. at least 96% sequence identity, e.g. at least 97% sequence identity, e.g. at least 98% sequence identity or at least 99% sequence identity to SEQ ID NO: 20, wherein the amino acid in position 44 is not a cysteine.

15. The bispecific antibody of claim 13 or 14, wherein the second scFv comprises or consists of the sequence of SEQ ID NO: 21, or comprising or consisting of a sequence having at least 90% sequence identity, e.g. at least 95% sequence identity, e.g. at least 96% sequence identity, e.g. at least 97% sequence identity, e.g. at least 98% sequence identity or at least 99% sequence identity to SEQ ID NO: 21.

16. The bispecific antibody of claim 12, wherein the second scFv is capable of binding CD38, and comprises 6 CDR sequences each consisting of the sequences of SEQ ID NO: 22-27 or sequences that differs by 1 or 2 substitutions from the sequences of SEQ ID NO: 22-27.

17. The bispecific antibody of claim 16, comprising
a. a VL sequence with the sequence of SEQ ID NO: 28 or a sequence having at least 90% sequence identity, e.g., at least 95% sequence identity, e.g. at least 96% sequence identity, e.g. at least 97% sequence identity, e.g. at least 98% sequence identity or at least 99% sequence identity to SEQ ID NO: 28, wherein the amino acid in position 100 Is not a cysteine; and
b. a VH sequence with the sequence of SEQ ID NO: 29 or a sequence having at least 90% sequence identity, e.g., at least 95% sequence identity, e.g. at least 96% sequence identity, e.g. at least 97% sequence identity, e.g. at least 98% sequence identity or at least 99% sequence identity to SEQ ID NO: 29, wherein the amino acid in position 44 is not a cysteine.

18. The bispecific antibody of claim 16 or 17, wherein the second scFv comprises or consists of the sequence of SEQ ID NO: 30, or comprising or consisting of a sequence having at least 90% sequence identity, e.g., at least 95% sequence identity, e.g., at least 96% sequence identity, e.g., at least 97% sequence identity, e.g., at least 98% sequence identity or at least 99% sequence identity to SEQ ID NO: 30.

19. The bispecific antibody of claim 12, wherein the second scFv is capable of binding CD20, and comprises 6 CDR sequences each consisting of the sequences of SEQ ID NO: 31-36 or sequences that differs by 1 or 2 substitutions from the sequences of SEQ ID NO: 31-36.

20. The bispecific antibody of claim 19, comprising
a. a VL sequence with the sequence of SEQ ID NO: 37 or a sequence having at least 90% sequence identity, e.g., at least 95% sequence identity, e.g., at least 96% sequence identity, e.g., at least 97% sequence identity, e.g., at least 98% sequence identity or at least 99% sequence identity to SEQ ID NO: 37, wherein the amino acid in position 99 is not a cysteine; and
b. a VH sequence with the sequence of SEQ ID NO: 38 or a sequence having at least 90% sequence identity, e.g., at least 95% sequence identity, e.g., at least 96% sequence identity, e.g., at least 97% sequence identity, e.g., at least 98% sequence identity or at least 99% sequence identity to SEQ ID NO: 38, wherein the amino acid in position 44 is not a cysteine.

21. The bispecific antibody of claim 19 or 20, wherein the second scFv comprises or consists of the sequence of SEQ ID NO: 39, or comprising or consisting of a sequence having at least 90% sequence identity, e.g., at least 95% sequence identity, e.g., at least 96% sequence identity, e.g., at least 97% sequence identity, e.g., at least 98% sequence identity or at least 99% sequence identity to SEQ ID NO: 39.

22. The bispecific antibody of claim 12, wherein the second scFv is capable of binding GPA33, and comprises 6 CDR sequences each consisting of the sequences of SEQ ID NO: 50-55 or sequences that differs by 1 or 2 substitutions from the sequences of SEQ ID NO: 50-55.

23. The bispecific antibody of claim 61, comprising
a. a VL sequence with the sequence of SEQ ID NO: 56 or a sequence having at least 90% sequence identity, e.g., at least 95% sequence identity, e.g., at least 96% sequence identity, e.g., at least 97% sequence identity, e.g., at least 98% sequence identity or at least 99% sequence identity to SEQ ID NO: 56, wherein the amino acid in position 44 is not a cysteine; and
b. a VH sequence with the sequence of SEQ ID NO: 57 or a sequence having at least 90% sequence identity, e.g., at least 95% sequence identity, e.g., at least 96% sequence identity, e.g., at least 97% sequence identity, e.g., at least 98% sequence identity or at least 99% sequence identity to SEQ ID NO: 57, wherein the amino acid in position 100 is not a cysteine.

24. The bispecific antibody of claim 61 or 62, wherein the second scFv comprises or consists of the sequence of SEQ ID NO: 61, or comprising or consisting of a sequence having at least 90% sequence identity, e.g., at least 95% sequence identity, e.g., at least 96% sequence identity, e.g., at least 97% sequence identity, e.g., at least 98% sequence identity or at least 99% sequence identity to SEQ ID NO: 61.

25. The bispecific antibody of claim 12, wherein the second scFv is capable of binding RSV, and comprises 6 CDR sequences consisting of amino acids 26-35, 53-59, 98-109, 177-181, 199-201 and 238-246 of SEQ ID NO: 62 or sequences that differ from these sequences by 1 or 2 substitutions.

26. The bispecific antibody of claim 25, comprising
a. a VL sequence with the sequence of amino acids 151-256 of SEQ ID NO: 62 or a sequence having at least 90% sequence identity, e.g., at least 95% sequence identity, e.g., at least 96% sequence identity, e.g., at least 97% sequence identity, e.g., at least 98% sequence identity or at least 99% sequence identity to amino acids 151-256 of SEQ ID NO: 62; and
b. a VH sequence with the sequence of amino acids 1-120 of SEQ ID NO: 62, or a sequence having at least 90% sequence identity, e.g., at least 95% sequence identity, e.g., at least 96% sequence identity, e.g., at least 97% sequence identity, e.g., at least 98% sequence identity or at least 99% sequence identity to amino acids 1-120 of SEQ ID NO: 62.

27. The bispecific antibody of claim 25 or 26, wherein the second scFv comprises or consists of the sequence of amino acids 1-256 of SEQ ID NO: 62, or comprising or consisting of a sequence having at least 90% sequence identity, e.g., at least 95% sequence identity, e.g., at least 96% sequence identity, e.g., at least 97% sequence identity, e.g., at least 98% sequence identity or at least 99% sequence identity to amino acids 1-256 of SEQ ID NO: 62.

28. The bispecific antibody of claim 12, wherein the second scFv is capable of binding B7H3, and comprises 6 CDR sequences consisting of amino acids 26-33, 51-58, 97-107, 175-180, 198-200 and 237-245 of SEQ ID NO: 63 or sequences that differ from these sequences by 1 or 2 substitutions.

29. The bispecific antibody of claim 28 comprising
a. a VL sequence with the sequence of amino acids 149-255 of SEQ ID NO: 63 or a sequence having at least 90% sequence identity, e.g., at least 95% sequence identity, e.g., at least 96% sequence identity, e.g., at least 97% sequence identity, e.g., at least 98% sequence identity or at least 99% sequence identity to amino acids 149-255 of SEQ ID NO: 63; and
b. a VH sequence with the sequence of amino acids 1-118 of SEQ ID NO: 63, or a sequence having at least 90% sequence identity, e.g., at least 95% sequence identity, e.g., at least 96% sequence identity, e.g., at least 97% sequence identity, e.g., at least 98% sequence identity or at least 99% sequence identity to amino acids 1-118 of SEQ ID NO: 63.

30. The bispecific antibody of claim 28 or 29, wherein the second scFv comprises or consists of the sequence of amino acids 1-255 of SEQ ID NO: 63, or comprising or consisting of a sequence having at least 90% sequence identity, e.g., at least 95% sequence identity, e.g., at least 96% sequence identity, e.g., at least 97% sequence identity, e.g., at least 98% sequence identity or at least 99% sequence identity to amino acids 1-255 of SEQ ID NO: 63.

31. The bispecific antibody of claim 12, wherein the scFv is capable of binding HER2, and comprises 6 CDR sequences consisting of amino acids 27-32, 50-52, 89-97, 164-171, 189-196 and 235-247 of SEQ ID NO: 64 or sequences that differ from these sequences by 1 or 2 substitutions.

32. The bispecific antibody of claim 31, comprising
a. a VL sequence with the sequence of amino acids 1-108 of SEQ ID NO: 64 or a sequence having at least 90% sequence identity, e.g., at least 95% sequence identity, e.g., at least 96% sequence identity, e.g., at least 97% sequence identity, e.g., at least 98% sequence identity or at least 99% sequence identity to amino acids 1-108 of SEQ ID NO: 64; and
b. a VH sequence with the sequence of amino acids 138-258 of SEQ ID NO: 64, or a sequence having at least 90% sequence identity, e.g., at least 95% sequence identity, e.g., at least 96% sequence identity, e.g., at least 97% sequence identity, e.g., at least 98% sequence identity or at least 99% sequence identity to amino acids 138-258 of SEQ ID NO: 64.

33. The bispecific antibody of claim 31 or 32, wherein the second scFv comprises or consists of the sequence of amino acids 1-258 of SEQ ID NO: 64 or amino acids 1-258 of SEQ ID NO: 65, or comprising or consisting of a sequence having at least 90% sequence identity, e.g., at least 95% sequence identity, e.g., at least 96% sequence identity, e.g., at least 97% sequence identity, e.g., at least 98% sequence identity or at least 99% sequence identity to amino acids 1-258 of SEQ ID NO: 64 or amino acids 1-258 of SEQ ID NO: 65.

34. The bispecific antibody of claim 12, wherein the scFv is capable of binding HER2, and comprises 6 CDR sequences consisting of amino acids 26-33, 51-58, 97-108, 176-181, 199-201 and 238-246 of SEQ ID NO: 66 or sequences that differ from these sequences by 1 or 2 substitutions.

35. The bispecific antibody of claim 34, comprising
a. a VL sequence with the sequence of amino acids 150-256 of SEQ ID NO: 66 or a sequence having at least 90% sequence identity, e.g., at least 95% sequence identity, e.g., at least 96% sequence identity, e.g., at least 97% sequence identity, e.g., at least 98% sequence identity or at least 99% sequence identity to amino acids 150-256 of SEQ ID NO: 66; and
b. a VH sequence with the sequence of amino acids 1-119 of SEQ ID NO: 66, or a sequence having at least 90% sequence identity, e.g., at least 95% sequence identity, e.g., at least 96% sequence identity, e.g., at least 97% sequence identity, e.g., at least 98% sequence identity or at least 99% sequence identity to amino acids 1-119 of SEQ ID NO: 66.

36. The bispecific antibody of claim 34 or 35, wherein the second scFv comprises or consists of the sequence of amino acids 1-256 of SEQ ID NO: 66 or amino acids 1-256 of SEQ ID NO: 67, or comprising or consisting of a sequence having at least 90% sequence identity, e.g., at least 95% sequence identity, e.g., at least 96% sequence identity, e.g., at least 97% sequence identity, e.g., at least 98% sequence identity or at least 99% sequence identity to amino acids 1-256 of SEQ ID NO: 66 or amino acids 1-256 of SEQ ID NO: 67.

37. The bispecific antibody according to any of the preceding claims, wherein the SADA domain is selected among domains comprising one of the sequences of SEQ ID NO: 5-12 or sequences that differs from one of SEQ ID NO: 5-12 by 1, 2, 3, 4, 5, 6, 7, 8, 9 or 10 substitutions.

38. The bispecific antibody according to any of the preceding claims, wherein the SADA domain comprises an amino acid sequence of amino acids 6-36 of SEQ ID NO: 5 or a sequence that differs from amino acids 6-36 of SEQ ID NO: 5 by one or more substitutions selected among:
E6V, Q, K, G, D or A;
Y7S, N, H, F, D or C;
F8Y, V, S, L, I or C;
T9S, P, N or A;
L10V, I or F;
Q11R, L, K, H or E;
I12V, T, M, L or F;
R13S, P, L, H, G or C;
G14W, R or A;
R15S, P, L, H, G or C;
E16V, Q, K, G, D or A;
F18Y, V, S, L, I or C;
E19V, Q, K, G, D or A;
M20V, T, R, L, K or I;
F21L or I;
R22L or G;
E23V, Q, K, G, D or A;
L24M;
N25S, I or D;
E26V, Q, K, G, D or A;
A27V, T, S, G or D;
L28W, V, M or F;
E29Q, G or D;
L30V, R, I, H or F;
K31T, R, Q, N, M or E;
D32Y, V, N, H, G or A;
A33V, T, S, P, G or D;
Q34R, L, K, H or E;
using the numbering of SEQ ID NO: 5.

39. The bispecific antibody according to any of the preceding claims, comprising or consisting of one of the sequences of SEQ ID NO: 40, 41, 42, 58, 59, 62, 63, 64, 65, 66, 67, 68 and 69.

40. A method of generating variants of a scFv domain, comprising a light chain variable domain (VL), a heavy chain variable domain (VH) and one or more disulfide bonds between the VL and the VH, comprising the steps of
a. Identifying the cysteine residues forming said one or more disulfide bonds between VL and VH; and
b. Substituting the cysteine residues forming one or more of said disulfide bonds identified in step a., with amino acids different from cysteine.

41. The method of claim 40, wherein said variants give rise to less multimer formation compared with said scFv domain.

42. The method of claim 41, wherein multimer formation is determined by SDS-PAGE gelelectrophoresis.

43. The method according to any of claims 40-42, wherein said scFv domain is part of a polypeptide comprising additional antibody fragments.

44. The method of any of claims 40-43, wherein said scFv domain is part of a bi- or a multispecific antibody.

45. The method of claim 44, wherein the bi- or multispecific antibody further comprises a SADA domain.

46. An scFv domain comprising a VL and a VH and capable of binding an antigen, wherein the scFv is obtainable according to the method of claims 40-45.

47. The scFv domain of claim 46, wherein the VH and VL are not connected by any disulfide bond.

48. The scFv domain according to claim 46 or 47, wherein the scFv further comprises a linker between the VH and VL.

49. The scFv domain according to any of claims 46 to 48, wherein the scFv is capable of binding DOTA-metal, and comprises 6 CDR sequences each consisting of the sequences of SEQ ID NO: 44-49 or sequences that differs by 1 or 2 substitutions from the sequences of SEQ ID NO: 44-49.

50. The scFv of claim 49, comprising
a. a VL sequence with the sequence of SEQ ID NO: 1 or a sequence having at least 90% sequence identity, e.g. at least 95% sequence identity, e.g. at least 96% sequence identity, e.g. at least 97% sequence identity, e.g. at least 98% sequence identity or at least 99% sequence identity to SEQ ID NO: 1, wherein the amino acid in position 111 is not a cysteine; and
b. a VH sequence with the sequence of SEQ ID NO: 2 or a sequence having at least 90% sequence identity, e.g. at least 95% sequence identity, e.g. at least 96% sequence identity, e.g. at least 97% sequence identity, e.g. at least 98% sequence identity or at least 99% sequence identity to SEQ ID NO: 2, wherein the amino acid in position 45 is not a cysteine.

51. The scFv of claim 49 or 50, comprising or consisting of the sequence of SEQ ID NO: 4, or comprising or consisting of a sequence having at least 90% sequence identity, e.g. at least 95% sequence identity, e.g. at least 96% sequence identity, e.g. at least 97% sequence identity, e.g. at least 98% sequence identity or at least 99% sequence identity to SEQ ID NO: 4.

52. The scFv domain according to any of the claims 46 to 48, wherein the scFv is capable of binding GD2, and comprises 6 CDR sequences each consisting of the sequences of SEQ ID NO: 13-18 or sequences that differs by 1 or 2 substitutions from the sequences of SEQ ID NO: 13-18.

53. The scFv of claim 52, comprising
a. a VL sequence with the sequence of SEQ ID NO: 19 or a sequence having at least 90% sequence identity, e.g. at least 95% sequence identity, e.g. at least 96% sequence identity, e.g. at least 97% sequence identity, e.g. at least 98% sequence identity or at least 99% sequence identity to SEQ ID NO: 19, wherein the amino acid in position 97 is not a cysteine; and
b. a VH sequence with the sequence of SEQ ID NO: 20 or a sequence having at least 90% sequence identity, e.g. at least 95% sequence identity, e.g. at least 96% sequence identity, e.g. at least 97% sequence identity, e.g. at least 98% sequence identity or at least 99% sequence identity to SEQ ID NO: 20, wherein the amino acid in position 44 is not a cysteine.

54. The scFv of claim 51 or 52, comprising or consisting of the sequence of SEQ ID NO: 21, or comprising or consisting of a sequence having at least 90% sequence identity, e.g. at least 95% sequence identity, e.g. at least 96% sequence identity, e.g. at least 97% sequence identity, e.g. at least 98% sequence identity or at least 99% sequence identity to SEQ ID NO: 21.

55. The scFv domain according to any of claims 46 to 48, wherein the scFv is capable of binding CD38, and comprises 6 CDR sequences each consisting of the sequences of SEQ ID NO: 22-27 or sequences that differs by 1 or 2 substitutions from the sequences of SEQ ID NO: 22-27.

56. The scFv of claim 55, comprising
a. a VL sequence with the sequence of SEQ ID NO: 28 or a sequence having at least 90% sequence identity, e.g., at least 95% sequence identity, e.g. at least 96% sequence identity, e.g. at least 97% sequence identity, e.g. at least 98% sequence identity or at least 99% sequence identity to SEQ ID NO: 28, wherein the amino acid in position 100 Is not a cysteine; and
b. A VH sequence with the sequence of SEQ ID NO: 29 or a sequence having at least 90% sequence identity, e.g., at least 95% sequence identity, e.g. at least 96% sequence identity, e.g. at least 97% sequence identity, e.g. at least 98% sequence identity or at least 99% sequence identity to SEQ ID NO: 29, wherein the amino acid in position 44 is not a cysteine.

57. The scFv of claim 55 or 56, comprising or consisting of the sequence of SEQ ID NO: 30, or comprising or consisting of a sequence having at least 90% sequence identity, e.g., at least 95% sequence identity, e.g., at least 96% sequence identity, e.g., at least 97% sequence identity, e.g., at least 98% sequence identity or at least 99% sequence identity to SEQ ID NO: 30.

58. The scFv domain according to any of claims 46 to 48, wherein the scFv is capable of binding CD20, and comprises 6 CDR sequences each consisting of the sequences of SEQ ID NO: 31-36 or sequences that differs by 1 or 2 substitutions from the sequences of SEQ ID NO: 31-36.

59. The scFv of claim 58, comprising
a. a VL sequence with the sequence of SEQ ID NO: 37 or a sequence having at least 90% sequence identity, e.g., at least 95% sequence identity, e.g., at least 96% sequence identity, e.g., at least 97% sequence identity, e.g., at least 98% sequence identity or at least 99% sequence identity to SEQ ID NO: 37, wherein the amino acid in position 99 is not a cysteine; and
b. a VH sequence with the sequence of SEQ ID NO: 38 or a sequence having at least 90% sequence identity, e.g., at least 95% sequence identity, e.g., at least 96% sequence identity, e.g., at least 97% sequence identity, e.g., at least 98% sequence identity or at least 99% sequence identity to SEQ ID NO: 38, wherein the amino acid in position 44 is not a cysteine.

60. The scFv of claim 58 or 59, comprising or consisting of the sequence of SEQ ID NO: 39, or comprising or consisting of a sequence having at least 90% sequence identity, e.g., at least 95% sequence identity, e.g., at least 96% sequence identity, e.g., at least 97% sequence identity, e.g., at least 98% sequence identity or at least 99% sequence identity to SEQ ID NO: 39.

61. The scFv domain according to any of claims 46 to 48, wherein the scFv is capable of binding GPA33, and comprises 6 CDR sequences each consisting of the sequences of SEQ ID NO: 50-55 or sequences that differs by 1 or 2 substitutions from the sequences of SEQ ID NO: 50-55.

62. The scFv of claim 61, comprising
a. a VL sequence with the sequence of SEQ ID NO: 56 or a sequence having at least 90% sequence identity, e.g., at least 95% sequence identity, e.g., at least 96% sequence identity, e.g., at least 97% sequence identity, e.g., at least 98% sequence identity or at least 99% sequence identity to SEQ ID NO: 56, wherein the amino acid in position 44 is not a cysteine; and
b. a VH sequence with the sequence of SEQ ID NO: 57 or a sequence having at least 90% sequence identity, e.g., at least 95% sequence identity, e.g., at least 96% sequence identity, e.g., at least 97% sequence identity, e.g., at least 98% sequence identity or at least 99% sequence identity to SEQ ID NO: 57, wherein the amino acid in position 100 is not a cysteine.

63. The scFv of claim 61 or 62, comprising or consisting of the sequence of SEQ ID NO: 61, or comprising or consisting of a sequence having at least 90% sequence identity, e.g., at least 95% sequence identity, e.g., at least 96% sequence identity, e.g., at least 97% sequence identity, e.g., at least 98% sequence identity or at least 99% sequence identity to SEQ ID NO: 61.

64. A composition comprising a bispecific antibody according to any of claims 1 to 39 or a scFv according to any of claims 46-63.

65. The composition of claim 64, being a pharmaceutical composition.

66. Use of a bispecific antibody according to any of claims 1 to 39, a scFv according to any of claims 46-63 or a composition according to claim 64 or 65, for diagnosing or treating cancer.

67. The use according to claim 66, in a method for treating or diagnosing cancer, comprising the steps:
a. Administering a bispecific antibody according to any of the claims 1-39, to a patient in need thereof; and
b. After a holding period administering a chelator binding a radionuclide.

68. The use according to claim 67, wherein the holding period is in the range of 24 hours to 96 hours.

69. The use according to any of claims 67-68, wherein the chelator is DOTA, DOTAM or a derivative
thereof selected among DOTA, Benzyl DOTA and the bischelate compound 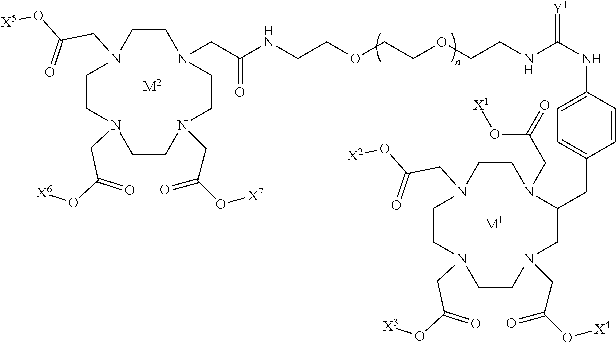
wherein X1, X2, X3, and X4 are each independently a lone pair of electrons (i.e. providing an oxygen anion) or H;
X5, X6, and X7 are each independently a lone pair of electrons (i.e. providing an oxygen anion) or H;
Y1 is O or S; and
n is 1, 2, 3, 4, 5, 6, 7, 8, 9, 10, 11, 12, 13, 14, 15, 16, 17, 18, 19, 20, 21, or 22; and
M1 is selected among is 175Lu3+, 45Sc3+, 69Ga3+, 71Ga3+, 89Y3+, 113In3+, 115In3+, 139La3+, 136Ce3+, 138Ce3+, 140Ce3+, 142Ce3+, 151Eu3+, 153Eu3+, 159Tb3+, 154Gd3+, 155Gd3+, 156Gd3+, 157Gd3+, 158Gd3+, or 160Gd3+;
M2 is selected among radionuclides.

70. The use according to any of claims 66-69, wherein the radionuclide is selected among ²¹¹At, ⁵¹Cr, ⁵⁷Co, ⁵⁸Co, ⁶⁷Cu, ¹⁵²Eu, ⁶⁷Ga, ¹¹¹In, ⁵⁹Fe, ²¹²Pb, ¹⁷⁷Lu, ²²³Ra, ²²⁴Ra, ¹⁸⁶Re, ¹⁸⁸Re, ⁷⁵Se, ^{99m}Tc, ²²⁷Th, ⁸⁹Zr, ⁹⁰Y, ^{94m}Tc, ⁶⁴Cu, ⁶⁸Ga, ⁶⁶Ga, ⁸⁶Y, ⁸²Rb, ^{110m}In, ²⁰⁹Bi, ²¹¹Bi, ²¹²Bi, ²¹³Bi, ²¹⁰Po, ²¹¹Po, ²¹²Po, ²¹⁴Po, ²¹⁵Po, ²¹⁶Po, ²¹⁸Po, ²¹¹At, ²¹⁵At, ²¹⁷At, ²¹⁸At, ²¹⁸Rn, ²¹⁹Rn, ²²⁰Rn, ²²²Rn, ²²⁶Rn, ²²¹Fr, ²²³Ra, ²²⁴Ra, ²²⁶Ra, ²²⁵Ac, ²²⁷Ac, ²²⁷Th, ²²⁸Th, ²²⁹Th, ²³⁰Th, ²³²Th, ²³¹Pa, ²³³U, ²³⁴U, ²³⁵U, ²³⁶U, ²³⁸U, ²³⁷Np, ²³⁸Pu, ²³⁹Pu, ²⁴⁰Pu, ²⁴⁴Pu, ²⁴¹Am, ²⁴⁴Cm, ²⁴⁵Cm, ²⁴⁸Cm, ²⁴⁹Cf, and ²⁵²Cf, preferable among ¹⁷⁷Lu, ^{99m}Tc, ⁶⁴Cu and ⁸⁹Zr.

71. The use according to any of claims 66 to 70, further comprising administering a clearing agent after step a., and before step b.

72. The use according to any of claims 66 to 71, further comprising detecting the localization of the radionuclide.

73. The use according to claim 72, wherein the radionuclide is detected using a PET or SPECT scanner.

74. The use according to any of claims 66 to 73, wherein the cancer is selected among osteosarcoma, liposarcoma, fibrosarcoma, malignant fibrous histiocytoma, leiomyosarcoma, spindle cell sarcoma, brain tumor, small cell lung cancer, retinoblastoma, HTLV-1 infected T cell leukemia.

75. The use according to any of the claim 66 to 74, further comprising a second and optional further administration of chelator binding a radionuclide.

76. A kit comprising a bispecific antibody according to any of claims 1 to 39.

77. The kit according to claim 76, further comprising a chelator that can be bound by the bispecific antibody.

78. The kit according to claim 76 or 77, further comprising instructions for use.

79. A polynucleotide encoding a bispecific antibody according to any of claims 1 to 39 or a scFv according to any of claims 46-63.

80. An expression vector or constructs comprising the polynucleotide of claim 79.

81. A host cell comprising the polynucleotide of claim 79 or the expression vector or construct of claim 80.

82. A method of producing a bispecific antibody according to any of claims 1-39 or a scFv according to any of claims 46-63, comprising the steps of
a. Providing a host cell of claim 81;
b. growing the host cell under conditions inducing expression of the polynucleotide; and
c. recovering the scFv or bispecific antibody from the growth broth.
